SEQUENCE LISTING (95 sequences)

SEQ ID NO: 1 - DNA, 51 bp, Homo sapiens
tctgatgtaccaacctcaccaacattacagaggcctactcatatgaaccaa

SEQ ID NO: 2 - DNA, 37 bp, Homo sapiens
tatgaaaaatatgccaaccttgtgttagatgttagca

SEQ ID NO: 3 - DNA, 22 bp, Artificial
  Synthetic DNA
tctgatgtaccaacctcaccaa

SEQ ID NO: 4 - DNA, 24 bp, Artificial
  Synthetic DNA
caacctcaccaacattacagaggc

SEQ ID NO: 5 - DNA, 25 bp, Artificial
  Synthetic DNA
acagaggcctactcatatgaaccaa

SEQ ID NO: 6 - DNA, 24 bp, Artificial
  Synthetic DNA
accaacattacagaggcctactca

SEQ ID NO: 7 - DNA, 24 bp, Artificial
  Synthetic DNA
gccaaccttgtgttagatgttagc

SEQ ID NO: 8 - DNA, 25 bp, Artificial
  Synthetic DNA
tatgaaaaatatgccaaccttgtgt

SEQ ID NO: 9 - DNA, 25 bp, Artificial
  Synthetic DNA
gccaaccttgtgttagatgttagca

SEQ ID NO: 10 - DNA, 1062 bp, Homo sapiens
ggctgattttttgtatttttagtagagacggggtttcaccgtgttagccagggtggtctcgatctcctgacctcgtgatccgcccaccttggcctctgggcgaggattacaggcgtgatccacctcacctggcctctccatctttttaactgcagtgtcagcggtgttccttgtcttctctgcagatgcaggcagcagaatatagtggttataggaacacaggtggaaaccctgtccaaagcaagggctatcgggtatcacctctgaccatccttcccattcatcctccaggtcaagtctggcaccatctttgacaacttcctcatcaccaacgatgaggcatacgctgaggagtttggcaacgagacgtggggcgtaacaaaggtgaggcctggtcctggtcctgatgtcgggggcgggcagggctggcagggggcaaggccctgaggtgtgtgctctgcctgcaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagg

SEQ ID NO: 11 - DNA, 700 bp, Homo sapiens
tatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaattatggagtatgtgtctgtggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaactta

SEQ ID NO: 12 - DNA, 605 bp, Homo sapiens
ttttctattataaaaaaagaacaattaggagttattaagcatttcttatacgtagaacacatttcattttactcctctttggagcaattcatactttcagtgtattttgaagtgatatatatgtattttattttttcagataaatcaaaccttctagtcttcagaacgaatggtgtttctgatgtaccaacctcaccaacattacagaggcctactcatatgaaccaaatggtgtttcacaaaatcagaaatgaagatttgatatttgtaagtcattagatactcattactgtcttttttgtccttttaaaacaacatctgttttcttgatttacattcatgtgacattggaattattttgttatatacaaatttagttgtgatttaaatatttttcttatgaaaaatatgccaaccttgtgttagatgttagcaaaattaattatcttaattatcctaagaatggaatcttaattttctttttggaaatttaaaaatgattcttcacaaatctcaaattaaaaattaaaaatctacaaagacctatatcgcaactcccaagttctcaagaaagtaagggaaattcaaggggttaaaaataaaga

SEQ ID NO: 13 - DNA, 611 bp, Homo sapiens
cacagcgcctgcgccagggactgggcgccgggtgcgagtggggcggggctcggagaggggcgaggcgcggggcgcggagaggggcggggccctgaccttgcgggccgacggctgcgcaggtgcccgcagtgcccaggggcggcgaggggcggggccagagtaggggctggctggatgagggcggggctccggcccgggtgggccgaagtctgaccctttttgtctcctagcctggatctccttggtgaccgctctgcatctagtgctgggcctcagcgccgtcctgggcctgctgctgctgaggtggcagtttcctgcacactacaggtaccgcccccgccaggcaggagactggcggtggaccaggtggagccgaaggcctgtaaacaggcattcttggttcgctctgtgaccccagatctccgtccaccgcccgtgcgcacctacggcttcgccacttcctgcacgtcacctctgggactcgccgcggctccttacactctaacacgcccactataccgcccacctcgaacagccccgcctcctgctgctcacctcggcgactaggccaccgtccacccttcagccaaactgcccactccacccccatc

SEQ ID NO: 14 - DNA, 19 bp, Artificial
  Synthetic DNA
ctccacagaaacatactcc

SEQ ID NO: 15 - DNA, 25 bp, Artificial
  Synthetic DNA
cacaaaatcagagatttgatatttg

SEQ ID NO: 16 - DNA, 26 bp, Artificial
  Synthetic DNA
cacaaaatcagaaatttgatatttgt

SEQ ID NO: 17 - DNA, 26 bp, Artificial
  Synthetic DNA
cacaaaatcaaagatttgatatttgt

SEQ ID NO: 18 - DNA, 25 bp, Artificial
  Synthetic DNA
ccaaatggtgttaatcagaaatgaa

SEQ ID NO: 19 - DNA, 24 bp, Artificial
  Synthetic DNA
ggtgtttcacttaatcagaaatga

SEQ ID NO: 20 - DNA, 23 bp, Artificial
  Synthetic DNA
gtgtttcacctaatcagaaatga

SEQ ID NO: 21 - DNA, 20 bp, Artificial
  Synthetic DNA
ctccttgtccgctcctcgtc

SEQ ID NO: 22 - DNA, 19 bp, Artificial
  Synthetic DNA
atcctccgacaattgtcct

SEQ ID NO: 23 - DNA, 18 bp, Artificial
  Synthetic DNA
gaaactgcttcctcagca

SEQ ID NO: 24 - DNA, 17 bp, Artificial
  Synthetic DNA
ggaaactgcaacctcag

SEQ ID NO: 25 - DNA, 26 bp, Artificial
  Synthetic DNA
gagcaagctttctcacaagcatttgg

SEQ ID NO: 26 - DNA, 28 bp, Artificial
  Synthetic DNA
ctgacacctagctgtgatcctgaaactg

SEQ ID NO: 27 - DNA, 22 bp, Artificial
  Synthetic DNA
cgtaacaaaggtgaggcctggt

SEQ ID NO: 28 - DNA, 23 bp, Artificial
  Synthetic DNA
ggcctctctacagctcgtccttg

SEQ ID NO: 29 - DNA, 22 bp, Artificial
  Synthetic DNA
ctcctagcctggatctccttgg

SEQ ID NO: 30 - DNA, 28 bp, Artificial
  Synthetic DNA
acagagcgaaccaagaatgcctgtttac

SEQ ID NO: 31 - DNA, 18 bp, Artificial
  Synthetic DNA
ctccacagacacatactc

SEQ ID NO: 32 - DNA, 16 bp, Artificial
  Synthetic DNA
cctcctcctctttgcg

SEQ ID NO: 33 - DNA, 16 bp, Artificial
  Synthetic DNA
aaactgccacctcagc

SEQ ID NO: 34 - DNA, 24 bp, Artificial
  Synthetic DNA
cacaaaatcagaaatgaagatttg

SEQ ID NO: 35 - DNA, 31 bp, Artificial
  Synthetic DNA
ttttttttttttctccacagacacatactcc

SEQ ID NO: 36 - DNA, 31 bp, Artificial
  Synthetic DNA
ttttttttttttctccacagaaacatactcc

SEQ ID NO: 37 - DNA, 28 bp, Artificial
  Synthetic DNA
ttttttttttttaaactgccacctcagc

SEQ ID NO: 38 - DNA, 29 bp, Artificial
  Synthetic DNA
ttttttttttttggaaactgcaacctcag

SEQ ID NO: 39 - DNA, 30 bp, Artificial
  Synthetic DNA
ttttttttttttgaaactgcttcctcagca

SEQ ID NO: 40 - DNA, 34 bp, Artificial
  Synthetic DNA
ttttttttttttctctttgcgtttcttgtcttct

SEQ ID NO: 41 - DNA, 32 bp, Artificial
  Synthetic DNA
ttttttttttttctccttgtccgctcctcgtc

SEQ ID NO: 42 - DNA, 33 bp, Artificial
  Synthetic DNA
ttttttttttttctcatcatccttgtcctctgc

SEQ ID NO: 43 - DNA, 31 bp, Artificial
  Synthetic DNA
ttttttttttttatcctccgacaattgtcct

SEQ ID NO: 44 - DNA, 44 bp, Artificial
  Synthetic DNA
tttttttttttttcacaaaatcagaaatgaagatttgatatttg

SEQ ID NO: 45 - DNA, 38 bp, Artificial
  Synthetic DNA
tttttttttttttcacaaaatcagagatttgatatttg

SEQ ID NO: 46 - DNA, 42 bp, Artificial
  Synthetic DNA
ttttttttttttttttcacaaaatcagaaatttgatatttgt

SEQ ID NO: 47 - DNA, 43 bp, Artificial
  Synthetic DNA
tttttttttttttgtttcacaaaatcaaagatttgatatttgt

SEQ ID NO: 48 - DNA, 38 bp, Artificial
  Synthetic DNA
tttttttttttttaatggtgtttcacaaaatcagaaat

SEQ ID NO: 49 - DNA, 38 bp, Artificial
  Synthetic DNA
tttttttttttttccaaatggtgttaatcagaaatgaa

SEQ ID NO: 50 - DNA, 38 bp, Artificial
  Synthetic DNA
ttttttttttttttggtgtttcacttaatcagaaatga

SEQ ID NO: 51 - DNA, 36 bp, Artificial
  Synthetic DNA
tttttttttttttgtgtttcacctaatcagaaatga

SEQ ID NO: 52 - DNA, 25 bp, Artificial
  Synthetic DNA
ctactcatatgaaccaaatggtgtt

SEQ ID NO: 53 - DNA, 21 bp, Artificial
  Synthetic DNA
gaggagcagagcagaggacaa

SEQ ID NO: 54 - DNA, 22 bp, Artificial
  Synthetic DNA
cagaggcttagaggaggcagag

SEQ ID NO: 55 - DNA, 20 bp, Artificial
  Synthetic DNA
gacgaggggcggacaaggag

SEQ ID NO: 56 - DNA, 75 bp, Artificial
  Synthetic DNA
gacgaggagcagaggcttaaggaggaggaagaagacaagaaacgcaaagaggaggaggaggcagaggacaaggag

SEQ ID NO: 57 - DNA, 23 bp, Artificial
  Synthetic DNA
gacgaggagcagaggacaaggag

SEQ ID NO: 58 - DNA, 29 bp, Artificial
  Synthetic DNA
gacgaggagcagaggcagaggacaaggag

SEQ ID NO: 59 - DNA, 41 bp, Artificial
  Synthetic DNA
gacgaggagcagaggcttaggaggaggcagaggacaaggag

SEQ ID NO: 60 - DNA, 23 bp, Artificial
  Synthetic DNA
gacgaggggcagaggacaaggag

SEQ ID NO: 61 - DNA, 33 bp, Artificial
  Synthetic DNA
ttttttttttttttgtcctctgctctgctcctc

SEQ ID NO: 62 - DNA, 34 bp, Artificial
  Synthetic DNA
ttttttttttttctctgcctcctctaagcctctg

SEQ ID NO: 63 - DNA, 32 bp, Artificial
  Synthetic DNA
ttttttttttttctccttgtccgcccctcgtc

SEQ ID NO: 64 - DNA, 16 bp, Artificial
  Synthetic DNA
gaggagcagaggcaga

SEQ ID NO: 65 - DNA, 16 bp, Artificial
  Synthetic DNA
gagcagaggcagagga

SEQ ID NO: 66 - DNA, 16 bp, Artificial
  Synthetic DNA
aggagcagaggcagag

SEQ ID NO: 67 - DNA, 17 bp, Artificial
  Synthetic DNA
aggagcagggcagagga

SEQ ID NO: 68 - DNA, 20 bp, Artificial
  Synthetic DNA
gaggagcaggcagaggacaa

SEQ ID NO: 69 - DNA, 21 bp, Artificial
  Synthetic DNA
acgaggagcagcagaggacaa

SEQ ID NO: 70 - DNA, 16 bp, Artificial
  Synthetic DNA
ggcttaggaggaggca

SEQ ID NO: 71 - DNA, 17 bp, Artificial
  Synthetic DNA
agaggcttaggaggagg

SEQ ID NO: 72 - DNA, 18 bp, Artificial
  Synthetic DNA
cagaggcttaggaggagg

SEQ ID NO: 73 - DNA, 20 bp, Artificial
  Synthetic DNA
cagaggcttagaggaggcag

SEQ ID NO: 74 - DNA, 22 bp, Artificial
  Synthetic DNA
gcagaggcttagaggaggcaga

SEQ ID NO: 75 - DNA, 20 bp, Artificial
  Synthetic DNA
gcagaggcttagaggaggca

SEQ ID NO: 76 - DNA, 22 bp, Artificial
  Synthetic DNA
agcagaggcttgaggaggcaga

SEQ ID NO: 77 - DNA, 21 bp, Artificial
  Synthetic DNA
gcagaggcttgaggaggcaga

SEQ ID NO: 78 - DNA, 19 bp, Artificial
  Synthetic DNA
gcagaggcttgaggaggca

SEQ ID NO: 79 - DNA, 20 bp, Artificial
  Synthetic DNA
gcagaggcttggaggaggca

SEQ ID NO: 80 - DNA, 21 bp, Artificial
  Synthetic DNA
agcagaggcttggaggaggca

SEQ ID NO: 81 - DNA, 23 bp, Artificial
  Synthetic DNA
agcagaggcttaaggaggcagag

SEQ ID NO: 82 - DNA, 22 bp, Artificial
  Synthetic DNA
agcagaggcttaaggaggcaga

SEQ ID NO: 83 - DNA, 16 bp, Artificial
  Synthetic DNA
ggggcagaggacaagg

SEQ ID NO: 84 - DNA, 15 bp, Artificial
  Synthetic DNA
gggcagaggacaagg

SEQ ID NO: 85 - DNA, 19 bp, Artificial
  Synthetic DNA
gacgaggggcggacaagga

SEQ ID NO: 86 - DNA, 19 bp, Artificial
  Synthetic DNA
acgaggggcggacaaggag

SEQ ID NO: 87 - DNA, 17 bp, Artificial
  Synthetic DNA
cgaggggcggacaagga

SEQ ID NO: 88 - DNA, 16 bp, Artificial
  Synthetic DNA
cgaggggcggacaagg

SEQ ID NO: 89 - DNA, 17 bp, Artificial
  Synthetic DNA
ggagcagaggacaagga

SEQ ID NO: 90 - DNA, 17 bp, Artificial
  Synthetic DNA
aggagcgaggacaagga

SEQ ID NO: 91 - DNA, 16 bp, Artificial
  Synthetic DNA
cgaggagcgaggacaa

SEQ ID NO: 92 - DNA, 23 bp, Artificial
  Synthetic DNA
gacgaggagcagaggacaaggag

SEQ ID NO: 93 - DNA, 22 bp, Artificial
  Synthetic DNA
gacgaggagcgaggacaaggag

SEQ ID NO: 94 - DNA, 21 bp, Artificial
  Synthetic DNA
gacgaggagcaggacaaggag

SEQ ID NO: 95 - DNA, 20 bp, Artificial
  Synthetic DNA
gacgaggagcggacaaggag

(19) United States
(12) Patent Application Publication
(10) Pub. No.: US 2022/0282314 A1
(43) Pub. Date: Sep. 8, 2022

(54) KIT FOR EVALUATING A GENE MUTATION RELATED TO MYELOPROLIFERATIVE NEOPLASMS

(71) Applicant: TOYO KOHAN CO., LTD., Tokyo (JP)

(72) Inventors: Emi TAKAMITSU, Kudamatsu-shi, Yamaguchi (JP); Midori YOSHIMURA, Kudamatsu-shi, Yamaguchi (JP); Mitsuyoshi OBA, Kudamatsu-shi, Yamaguchi (JP)

(73) Assignee: TOYO KOHAN CO., LTD., Tokyo (JP)

(21) Appl. No.: 17/637,642

(22) PCT Filed: Aug. 28, 2020
(86) PCT No.: PCT/JP2020/032584

§ 371 Date: Feb. 23, 2022

(30) Foreign Application Priority Data
Aug. 30, 2019 (JP) 2019-158722
Sep. 27, 2019 (JP) 2019-176891
Aug. 6, 2020 (JP) 2020-133602

(51) Int. Cl. C12Q 1/6827; C12Q 1/6837; C12Q 1/686; C12Q 1/6886

(52) U.S. Cl. CPC C12Q 1/6827; C12Q 1/6837; C12Q 1/686; C12Q 1/6886; C12Q 2600/156

(57) ABSTRACT

This invention is intended to accurately determine the presence or absence of a plurality of types of gene mutations in CALR or JAK2 among gene mutations related to myeloproliferative neoplasms. A CALR mutation probe reacts with any of the type 1, type 3, type 4, or the type 5 mutation related to myeloproliferative neoplasms and has a mismatch resulting from artificial deletion. Probes for JAK2 mutation encompass a V617F mutation probe and an exon 12 mutation probe.

### TECHNICAL FIELD

The present invention relates to a set of probes that enables evaluating a gene mutation as a useful diagnostic component of myeloproliferative neoplasms and microarrays comprising such set of probes.

### BACKGROUND ART

Myeloproliferative neoplasms (MPN) is a disease developed upon tumorigenesis of bone marrow cells. MPN is characterized by significant proliferation of bone marrow cells, such as blood granulocytes, gemmules, megakaryocytes, and mastocytes. MPN includes chronic myelogenous leukemia (CML), chronic neutrophilic leukemia (CNL), polycythemia vera (PV), primary myelofibrosis (PMF), essential thrombocythemia (ET), chronic eosinophilic leukemia (CEL), hypereosinophilic syndrome (HES), mastocytosis, and myeloproliferative neoplasms, unclassifiable (MPN, U).

As described in Non-Patent Document 1, MPN is diagnosed using, as indicators, clinical parameters, bone marrow configuration, and gene mutation data. A Philadelphia chromosome-negative patient may be subjected to diagnosis by employing the techniques described above in combination, so that MPN except for CML can be diagnosed. Specific examples of gene mutation data that may be employed include information on mutation of 3 genes (i.e., JAK2, CALR, and MPL) and additional mutation information on ASXL1, EZH2, TET2, IDH1/IDH2, SRSF2, and SF3B1). In particular, JAK2, CALR, and MPL are considered to be molecular bases for development of MPN. Accordingly, the presence or absence of mutation in such genes is an important element for definite diagnosis of MPN.

Non-Patent Document 2 discloses that, concerning JAK2, the JAK2 V617F mutation (i.e., the substitution of valine 617 with phenylalanine) is often observed in PV, ET, and PMF and that insertion-deletion mutation in exon 12 is observed in a small number of PV cases in addition to the above mutation. JAK2 (Janus activating kinase 2) is a gene encoding a protein that controls signaling of an erythropoietin receptor. Further, Non-Patent Document 3 discloses, concerning JAK2, a mutation in exon 12 is correlated with polycythemia vera (PV) or idiopathic erythrocytosis (IE). Further, Patent Document 5 discloses that the c2035t mutation (T514M mutation) is to be detected from among mutations existing in exon 12 of the JAK2 gene as a mutation indicating a myeloproliferative disorder.

Non-Patent Document 2 discloses that, concerning MPL, MPLW515L/K-mutated PMF is observed in PMF and ET. MPL is a gene encoding a thrombopoietin receptor.

Non-Patent Document 2 discloses that, concerning CALR, a 52-bp deletion type 1 mutation and a 5-bp insertion type 2 mutation are most frequently observed in the case of ET and PMF. Non-Patent Document 2 also discloses that the type 1 mutation is more frequently observed in PMF and is related to development of ET into myelofibrosis. CALR is a gene encoding calreticulin, which is an endoplasmic molecular chaperone.

Further, Patent Document 1 discloses, as a method for analysis of JAK2 gene mutation, a JAK2 V617F-site-specific fluorescence-labeled probe. Patent Document 2 discloses a technique of detecting a mutation different from the JAK2 V617F mutation, which was found in a JAK2-V617F-mutation-negative patient having myeloproliferative neoplasms.

Furthermore, Patent Document 3 discloses, as probes for detecting the MPL gene polymorphism, a set of probes for detecting the W515K mutation and the W515L mutation in MPL.

Furthermore, Patent Document 4 discloses a technique for identifying a CALR mutation.

Furthermore, Patent Document 5 discloses detection of a mutation in the JAK2 nucleic acid and a gene mutation in exon 12 in JAK2. Furthermore, Patent Document 6 discloses that primers and probes were designed for relevant gene mutations as means for simultaneously and readily detecting a plurality of gene mutations related to myeloproliferative neoplasms to dissolve the problems described above. In Patent Document 6, the V617F mutation in JAK2, the type 1 mutation and the type 2 mutation in CALR, and the W515L mutation and the W515K mutation in MPL are disclosed as gene mutations to be detected (5 gene mutations in 3 genes).

A probe for detecting a target gene mutation is designed based on a nucleotide sequence of a peripheral region including the target gene mutation. As disclosed in Patent Document 7, a probe is designed as a sequence that perfectly matches with the nucleotide sequence of a peripheral region including the target gene mutation or a nucleotide sequence containing one or several non-natural nucleotides. A probe that comprises one or several non-natural nucleotides does not form hybrids with a peripheral region including the target gene mutation at the position of the non-natural nucleotides (mismatch). According to Patent Document 7, whether or not the target nucleic acid in the sample has a gene mutation can be detected with high accuracy on the basis of such mismatch.

### PRIOR ART DOCUMENTS

### Patent Documents

Patent Document 1: JP 2012-034580 A
Patent Document 2: WO 2009/060804
Patent Document 3: WO 2011/052755
Patent Document 4: JP 2016-537012 A
Patent Document 5: JP Patent No. 6017136
Patent Document 6: WO 2019/004334
Patent Document 7: JP 2000-511434 A

### Non-Patent Documents

Non-Patent Document 1: Francesco Passamonti and Margherita Maffioli, Hematology 2016, pp. 534-542
Non-Patent Document 2: NCCN Clinical Practice Guidelines in Oncology (NCCN Guidelines), Myeloproliferative Neoplasms, Version 2, 2017, Oct. 19, 2016
Non-Patent Document 3: Linda M. Scott et al., N. Engl. J. Med., 2007 Feb. 1; 356 (5): 459-68

### SUMMARY OF THE INVENTION

### Objects to be Attained by the Invention

The present invention provides a kit for evaluating a gene mutation that enables accurate evaluation of the presence or absence of a plurality of types of gene mutations in CALR among gene mutations related to myeloproliferative neoplasms and enables evaluation of the presence or absence of myeloproliferative neoplasms with higher accuracy. More specifically, the present invention provides a kit for evaluating a gene mutation that enables simultaneous evaluation of the presence or absence of a plurality of types of gene mutations in JAK2 among gene mutations related to myeloproliferative neoplasms and enables evaluation of the presence or absence of myeloproliferative neoplasms with higher accuracy.

### Means for Attaining the Objects

The present invention include the following.

(1) A kit for evaluating a gene mutation related to myeloproliferative neoplasms comprising a CALR mutation probe corresponding to the gene mutation related to myeloproliferative neoplasms in CALR, which is at least 1 gene mutation selected from the group consisting of: a 52-bp deletion type 1 mutation resulting from deletion of 52 nucleotides at positions 506 to 557 in the nucleotide sequence of the wild-type CALR gene represented by SEQ ID NO: 10; a 46-bp deletion type 3 mutation resulting from deletion of 46 nucleotides at positions 509 to 554 in the nucleotide sequence represented by SEQ ID NO: 10; a 34-bp deletion type 4 mutation resulting from deletion of 34 nucleotides at positions 516 to 549 in the nucleotide sequence represented by SEQ ID NO: 10; and a 52-bp deletion type 5 mutation resulting from deletion of 52 nucleotides at positions 505 to 556 in the nucleotide sequence represented by SEQ ID NO: 10, wherein the CALR mutation probe comprises a mismatch caused by artificial deletion.
(2) The kit for evaluating a gene mutation according to (1), wherein

the CALR mutation probe corresponding to the type 1 mutation comprises anucleotide sequence derived from the nucleotide sequence represented by SEQ ID NO: 10 by deletion of 1 or a plurality of nucleotides selected from a region of 558 to 564 or a nucleotide sequence complementary thereto,

the CALR mutation probe corresponding to the type 3 mutation comprises anucleotide sequence derived from the nucleotide sequence represented by SEQ ID NO: 10 by deletion of 1 or a plurality of nucleotides selected from a region of 555 to 559 or a nucleotide sequence complementary thereto,

the CALR mutation probe corresponding to the type 4 mutation comprises anucleotide sequence derived from the nucleotide sequence represented by SEQ ID NO: 10 by deletion of 1 or a plurality of nucleotides selected from a region of 550 to 558 or a nucleotide sequence complementary thereto, and

the CALR mutation probe corresponding to the type 5 mutation comprises anucleotide sequence derived from the nucleotide sequence represented by SEQ ID NO: 10 by deletion of 1 or a plurality of nucleotides selected from a region of 558 to 564 or a nucleotide sequence complementary thereto.

(3) The kit for evaluating a gene mutation according to (1), wherein the CALR mutation probe corresponding to the type 1 mutation comprises the nucleotide sequence represented by SEQ ID NO: 95 or a nucleotide sequence complementary thereto, the CALR mutation probe corresponding to the type 3 mutation comprises the nucleotide sequence represented by SEQ ID NO: 53 or a nucleotide sequence complementary thereto, the CALR mutation probe corresponding to the type 4 mutation comprises the nucleotide sequence represented by SEQ ID NO: 54 or a nucleotide sequence complementary thereto, and the CALR mutation probe corresponding to the type 5 mutation comprises the nucleotide sequence represented by SEQ ID NO: 55 or a nucleotide sequence complementary thereto.
(4) The kit for evaluating a gene mutation according to (1), which further comprises a CALR mutation probe corresponding to the type 2 mutation resulting from insertion of TTGTC between positions 568 and 569 in the nucleotide sequence of the wild-type CALR gene represented by SEQ ID NO: 10.
(5) The kit for evaluating a gene mutation according to (1), which further comprises a JAK2 mutation probe corresponding to the gene mutation related to myeloproliferative neoplasms in JAK2 and/or an MPL mutation probe corresponding to the gene mutation related to myeloproliferative neoplasms in MPL.
(6) A method of analyzing data concerning diagnosis of myeloproliferative neoplasms, comprising identifying at least 1 gene mutation selected from the group consisting of the type 1 mutation, the type 3 mutation, the type 4 mutation, and the type 5 mutation related to myeloproliferative neoplasms in CALR using the kit for evaluating a gene mutation according to any of (1) to (5) in a target of diagnosis.
(7) A kit for evaluating a gene mutation related to myeloproliferative neoplasms comprising JAK2 mutation probes corresponding to the gene mutation related to myeloproliferative neoplasms in JAK2 and a set of primers that amplifies a region including the gene mutation, wherein the JAK2 mutation probes comprise a V617F mutation probe corresponding to the V617F mutation and an exon 12 mutation probe corresponding to a gene mutation existing in exon 12 of the JAK2 gene, and the set of primers comprises a set of primers for the V617F mutation that amplifies a region including the V617F mutation and a set of primers for exon 12 that amplifies a region including the gene mutation existing in exon 12 of the JAK2 gene.
(8) The kit for evaluating a gene mutation according to (7), wherein the exon 12 mutation probe is at least 1 mutation probe selected from the group consisting of a N542_E543del mutation probe corresponding to a deletion mutation of N542-E543 in JAK2, a E543_D544del mutation probe corresponding to a deletion mutation of E543-D544 in JAK2, a R541_E543>K mutation probe corresponding to a substitution mutation of R541-E543 with lysine in JAK2, a F537_K539>L mutation probe corresponding to a substitution mutation of F537-K539 with leucine in JAK2, a K539L (TT) mutation probe corresponding to a mutation of K539L (TT) in JAK2, and a K539L (CT) mutation probe corresponding to a mutation of K539L (CT) in JAK2.
(9) The kit for evaluating a gene mutation according to (7), wherein the concentration of a primer included in the set of primers for the V617F mutation is 1.0 μM or higher.
(10) The kit for evaluating a gene mutation according to (7), wherein the concentration of a primer included in the set of primers for exon 12 is 2.5 μM or higher.
(11) The kit for evaluating a gene mutation according to (7), wherein the ratio of the concentration of the labeled primer of the set of primers for the V617F mutation to the concentration of the labeled primer of the set of primers for exon 12; [the concentration of the primer for exon 12]/[the concentration of the primer for the V617F mutation] is 1.0 to 5.5.
(12) The kit for evaluating a gene mutation according to (7), wherein the set of primers for exon 12 consists of a forward primer for exon 12 having 10 or more continuous nucleotides selected from the nucleotide sequence represented by SEQ ID NO: 1 and reverse primer for exon 12 having 10 or more continuous nucleotides selected from the nucleotide sequence represented by SEQ ID NO: 2.
(13) The kit for evaluating a gene mutation according to (12), wherein the forward primer for exon 12 is a primer selected from the group consisting of a forward primer F1 for exon 12 comprising the nucleotide sequence represented by SEQ ID NO: 3, a forward primer F3 for exon 12 comprising the nucleotide sequence represented by SEQ ID NO: 4, a forward primer F4 for exon 12 comprising the nucleotide sequence represented by SEQ ID NO: 5, and a forward primer F5 for exon 12 comprising the nucleotide sequence represented by SEQ ID NO: 6.
(14) The kit for evaluating a gene mutation according to (12), wherein the reverse primer for exon 12 is a primer selected from the group consisting of a reverse primer R1 for exon 12 comprising the nucleotide sequence represented by SEQ ID NO: 7, a reverse primer R2 for exon 12 comprising the nucleotide sequence represented by SEQ ID NO: 8, and a reverse primer R3 for exon 12 comprising the nucleotide sequence represented by SEQ ID NO: 9.
(15) The kit for evaluating a gene mutation according to (12), wherein the set of primers for exon 12 consists of the forward primer F5 for exon 12 comprising the nucleotide sequence represented by SEQ ID NO: 6 and the reverse primer R2 for exon 12 comprising the nucleotide sequence represented by SEQ ID NO: 8.
(16) The kit for evaluating a gene mutation according to (7), which further comprises:

a CALR mutation probe corresponding to the gene mutation related to myeloproliferative neoplasms in CALR;

a set of primers for CALR for amplifying a region including the gene mutation related to myeloproliferative neoplasms in CALR;

an MPL mutation probe corresponding to the gene mutation related to myeloproliferative neoplasms in MPL; and

a set of primers for MPL for amplifying a region including the gene mutation related to myeloproliferative neoplasms in MPL.

(17) The kit for evaluating a gene mutation according to (7), which comprises a microarray having the V617F mutation probe and the exon 12 mutation probe fixed on a support.
(18) A method of analyzing data concerning diagnosis of myeloproliferative neoplasms, comprising simultaneously identifying the V617F mutation and the gene mutation in exon 12 from among the JAK2 gene mutations related to myeloproliferative neoplasms using the kit for evaluating a gene mutation according to any of (7) to (17) in a target of diagnosis.

This description includes part or all of the content as disclosed in the descriptions and/or drawings of Japanese Patent Application Nos. 2019-158722, 2019-176891, and 2020-133602, which are priority documents of the present application.

### Effects of the Invention

According to the present invention, a plurality of gene mutations existing, in particular, in CALR (i.e., the type 1 mutation and the type 3 mutation to the type 5 mutation) from among gene mutations related to myeloproliferative neoplasms can be accurately determined. Accordingly, the present invention can improve accuracy for diagnosis of myeloproliferative neoplasms performed with the use of information on the gene mutation in the target of diagnosis.

According to the present invention, also, among gene mutations related to myeloproliferative neoplasms, a plurality of gene mutations existing, in particular, in JAK2 (i.e., the V617F mutation and the gene mutation in exon 12) can be simultaneously identified. According to the present invention, therefore, accuracy for diagnosis of myeloproliferative neoplasms performed with the use of information on the gene mutation in the target of diagnosis can be improved.

### BRIEF DESCRIPTION OF THE DRAWINGS

FIG. 1 shows a constitutional diagram illustrating deletion regions of the type 1 mutation, the type 3 mutation, the type 4 mutation, and the type 5 mutation in CALR.

FIG. 2 shows a characteristic diagram demonstrating the results of measurements of mutant samples and wild-type samples using the type 3 mutation probe 5, the type 4 mutation probe 5, and the type 5 mutation probe 4 designed in the examples.

FIG. 3 shows a constitutional diagram illustrating primers designed to amplify regions including a plurality of gene mutations in exon 12 in JAK2.

FIG. 4 shows a characteristic diagram demonstrating the results of measurements of fluorescence intensities derived from the amplified 4 fragments obtained with the use of the set of primers designed in Example 1.

FIG. 5 shows a characteristic diagram demonstrating, on the horizontal axis, the concentration of the labeled primer (the forward primer) in the set of primers that amplifies a region including the V617F mutation and, on the vertical axis, the fluorescence intensities derived from the amplified 4 regions.

FIG. 6 shows a characteristic diagram demonstrating, on the horizontal axis, the concentration of the labeled primer (the forward primer) in the set of primers that amplifies a region including the V617F mutation and, on the vertical axis, the fluorescence intensities derived from the amplified 4 regions.

FIG. 7 shows a characteristic diagram demonstrating, on the horizontal axis, the concentration of the labeled primer (the reverse primer) in the set of primers that amplifies exon 12 in JAK2 and, on the vertical axis, the fluorescence intensities derived from the amplified 4 regions.

FIG. 8 shows a characteristic diagram demonstrating, on the horizontal axis, the concentration of the labeled primer (the reverse primer) in the set of primers that amplifies exon 12 in JAK2 and, on the vertical axis, the fluorescence intensities derived from the amplified 4 regions.

FIG. 9 shows a characteristic diagram demonstrating, on the horizontal axis, the ratio of the concentration of the labeled primer (the reverse primer) of the set of primers that amplifies exon 12 to the concentration of the labeled primer (the forward primer) of the set of primers that amplifies a region including the V617F mutation and, on the vertical axis, the fluorescence intensities derived from the amplified 4 regions.

FIG. 10 shows a characteristic diagram demonstrating the results of detection of the V617F mutation, the gene mutation in exon 12 in JAK2 and so on using the mutation model analytes.

### EMBODIMENTS OF THE INVENTION

### <CALR Gene Mutation>

The kit for evaluating a gene mutation related to myeloproliferative neoplasms according to the present invention comprises a CALR mutation probe that identifies at least 1 gene mutation selected from the group consisting of the type 1 mutation, the type 3 mutation, the type 4 mutation, and the type 5 mutation as a gene mutation related to myeloproliferative neoplasms in CALR.

As CALR gene mutations, a 52-bp deletion type 1 mutation, a 5-bp insertion type 2 mutation, a 46-bp deletion type 3 mutation, a 34-bp deletion type 4 mutation, and another 52-bp deletion mutation different from the type 1 mutation; i.e., the type 5 mutation, are mainly known. The type 1 mutation to the type 5 mutation are each located at the C terminus of the CALR protein. Any of such mutations is observed in patients with primary myelofibrosis (PMF) or essential thrombocythemia (ET) at a frequency of 20% to 25%. The type 2 mutation is primarily related to essential thrombocythemia (ET) and the type 1 mutation is primarily related to primary myelofibrosis (PMF). CALR gene mutation is also observed in the case of myeloproliferative neoplasms without JAK2 gene mutation, which is described in detail below.

SEQ ID NO: 10 shows a nucleotide sequence encoding wild-type CALR. In the presence of the type 1 mutation, 52 nucleotides at positions 506 to 557 are deleted from the nucleotide sequence represented by SEQ ID NO: 10. In the presence of the type 2 mutation, the sequence TTGTC is inserted between nucleotides 568 and 569 in the nucleotide sequence represented by SEQ ID NO: 10. In the presence of the type 3 mutation, 46 nucleotides at positions 509 to 554 are deleted from the nucleotide sequence represented by SEQ ID NO: 10. In the presence of the type 4 mutation, 34 nucleotides at positions 516 to 549 are deleted from the nucleotide sequence represented by SEQ ID NO: 10. In the presence of the type 5 mutation, 52 nucleotides at positions 505 to 556 are deleted from the nucleotide sequence represented by SEQ ID NO: 10.

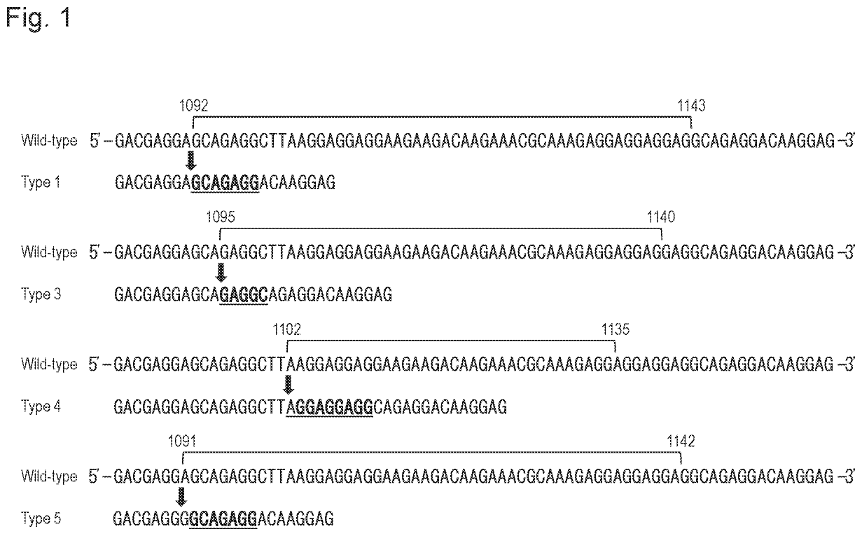

Concerning deletion-type gene mutations; i.e., the type 1 mutation, the type 3 mutation, the type 4 mutation, and the type 5 mutation, FIG. 1 schematically shows regions deleted from a part (SEQ ID NO: 56) of the nucleotide sequence encoding wild-type CALR. As shown in FIG. 1, the type 1 mutation lacking 52 nucleotides from wild-type CALR (SEQ ID NO: 57), the type 3 mutation lacking 46 nucleotides from wild-type CALR (SEQ ID NO: 58), the type 4 mutation lacking 34 nucleotides from wild-type CALR (SEQ ID NO: 59), and the type 5 mutation lacking 52 nucleotides from wild-type CALR (SEQ ID NO: 60) each have sequences very similar before and after deletion toward the 3′ terminus from the site of deletion (indicated by an arrow). In FIG. 1, the sequences consistent with each other before and after deletion in the nucleotide sequences of the type 1 mutation, the type 3 mutation, the type 4 mutation, and the type 5 mutation are underlined.

The kit for evaluating a gene mutation according to the present invention comprises, as a CALR mutation probe, at least 1 probe selected from the group consisting of a type 1 mutation probe detecting the type 1 mutation, a type 3 mutation probe detecting the type 3 mutation, a type 4 mutation probe detecting the type 4 mutation, and a type 5 mutation probe detecting the type 5 mutation. Specifically, the kit for evaluating a gene mutation according to the present invention may comprise all of the type 1 mutation probe, the type 3 mutation probe, the type 4 mutation probe, and the type 5 mutation probe or it may comprise 1 or 2 probes selected from among the type 3 mutation probe, the type 4 mutation probe, and the type 5 mutation probe.

Such CALR mutation probe has mismatches caused by artificial deletion. Specifically, a CALR mutation probe is designed as a complementary strand resulting from deletion of at least 1 nucleotide (1 to several nucleotides, such as 1 to 5 nucleotides, preferably 1 to 3 nucleotides, and more preferably 1 nucleotide) (i.e., artificial deletion) from the sequence resulting from the deletion mutation (i.e., the type 1 mutation, the type 3 mutation, the type 4 mutation, or the type 5 mutation) shown in FIG. 1. A nucleotide (or nucleotides) to be artificially deleted is(are) preferably selected from a region consistent with the wild-type sequence (the underlined region in FIG. 1) in the sequence after deletion mutation (i.e., the type 1 mutation, the type 3 mutation, the type 4 mutation, or the type 5 mutation) as shown in FIG. 1. While a CALR mutation probe can be designed as a strand complementary to a given nucleotide sequence, it may be designed as the same strand as the given nucleotide sequence.

Specifically, a CALR mutation probe corresponding to the type 1 mutation, the type 3 mutation, the type 4 mutation, or the type 5 mutation can be designed as a complementary strand lacking at least 1 nucleotide in a region of up to 10 nucleotides, preferably up to 8 nucleotides, and more preferably up to 5 nucleotides toward the 3′ terminus from the site of deletion (indicated by an arrow in FIG. 1).

More specifically, a CALR mutation probe corresponding to the type 1 mutation can be designed as a complementary strand lacking at least 1 nucleotide from a region of up to 7 nucleotides (underlined in FIG. 1) toward the 3′ terminus from the site of deletion (indicated by an arrow in FIG. 1). With reference to the nucleotide sequence represented by SEQ ID NO: 10, a CALR mutation probe corresponding to the type 1 mutation can be designed as a complementary strand lacking at least 1 nucleotide from a region of 7 nucleotides at positions 558 to 564. It is particularly preferable that a CALR mutation probe corresponding to the type mutation be designed as a complementary strand of the sequence GACGAGGAGCGGACAAGGAG (SEQ ID NO: 95) lacking AGA at positions 560 to 562 from the nucleotide sequence represented by SEQ ID NO: 10 (it may be designed as the nucleotide sequence represented by SEQ ID NO: 95).

A CALR mutation probe corresponding to the type 3 mutation can be designed as a complementary strand lacking at least 1 nucleotide from a region of 5 nucleotides (underlined in FIG. 1) toward the 3′ terminus from the site of deletion (indicated by an arrow in FIG. 1). With reference to the nucleotide sequence represented by SEQ ID NO: 10, a CALR mutation probe corresponding to the type 3 mutation can be designed as a complementary strand lacking at least 1 nucleotide from a region of 5 nucleotides at positions 555 to 559. It is particularly preferable that a CALR mutation probe corresponding to the type 3 mutation be designed as a complementary strand of the sequence GAGGAGCAGAGCAGAGGACAA (SEQ ID NO: 53) lacking G at position 558 from the nucleotide sequence represented by SEQ ID NO: 10 (it may be designed as the nucleotide sequence represented by SEQ ID NO: 53).

A CALR mutation probe corresponding to the type 4 mutation can be designed as a complementary strand lacking at least 1 nucleotide from a region of 9 nucleotides (underlined in FIG. 1) toward the 3′ terminus from the site of deletion (indicated by an arrow in FIG. 1). With reference to the nucleotide sequence represented by SEQ ID NO: 10, a CALR mutation probe corresponding to the type 4 mutation can be designed as a complementary strand lacking at least 1 nucleotide from a region of 9 nucleotides at position 550 to 558. It is particularly preferable that a CALR mutation probe corresponding to the type 4 mutation be designed as a complementary strand of the sequence CAGAGGCTTAGAGGAGGCAGAG (SEQ ID NO: 54) lacking G at position 552 from the nucleotide sequence represented by SEQ ID NO: 10 (it may be the nucleotide sequence represented by SEQ ID NO: 54).

A CALR mutation probe corresponding to the type 5 mutation can be designed as a complementary strand lacking at least 1 nucleotide from a region of 7 nucleotides at position 2 to 8 (underlined in FIG. 1) toward the 3′ terminus from the site of deletion (indicated by an arrow in FIG. 1). With reference to the nucleotide sequence represented by SEQ ID NO: 10, a CALR mutation probe corresponding to the type 5 mutation can be designed as a complementary strand lacking at least 1 nucleotide from a region of 7 nucleotides at positions 558 to 564. It is particularly preferable that a CALR mutation probe corresponding to the type 5 mutation be designed as a complementary strand of the sequence GACGAGGGGCGGACAAGGAG (SEQ ID NO: 55) lacking AGA at positions 560 to 562 from the nucleotide sequence represented by SEQ ID NO: 10 (it may be designed as the nucleotide sequence represented by SEQ ID NO: 55).

### <JAK2 Gene Mutation>

The kit for evaluating a gene mutation related to myeloproliferative neoplasms according to the present invention is intended to simultaneously identify the V617F mutation and the gene mutation in exon 12 as the gene mutations related to myeloproliferative neoplasms in JAK2. Specifically, the kit for evaluating a gene mutation comprises, as JAK2 mutation probes, a V617F mutation probe corresponding to the V617F mutation, which is the gene mutation related to myeloproliferative neoplasms in JAK2, and an exon 12 mutation probe corresponding to the gene mutation in exon 12, which is the gene mutation related to myeloproliferative neoplasms in JAK2. The kit for evaluating a gene mutation also comprises a set of primers for the V617F mutation that amplifies a region including the V617F mutation in JAK2 and a set of primers for exon 12 that amplifies a region including the gene mutation in exon 12 in JAK2.

Specifically, the JAK2 V617F gene mutation is substitution of valine 617 with phenylalanine. This substitution mutation contributes to activation of the JAK-STAT pathway and it is a significant feature in polycythemia vera (PV). Such V617F mutation is also observed in patients with primary myelofibrosis (PMF) or essential thrombocythemia (ET) at a frequency of 50% to 60%. SEQ ID NO: 11 shows the nucleotide sequence of exon 14 including valine 617 of the wild-type JAK2 gene. In the presence of the V617F mutation, G at position 351 is substituted with T in the nucleotide sequence as shown in SEQ ID NO: 11.

The gene mutation in exon 12 is known as the diagnostic criteria for MPN defined by World Health Organization (WHO), and, in particular, such gene mutation is detected in polycythemia vera (PV). Examples of JAK2 exon 12 gene mutations include, but are not particularly limited to, deletion of asparagine 542 and glutamic acid 543 (referred to as N542_E543del mutation), deletion of glutamic acid 543 and aspartic acid 544 (referred to as E543_D544del mutation), substitution of arginine 541 to glutamic acid 543 with lysine (referred to as R541_E543>K mutation), substitution of phenylalanine 537 to lysine 539 with leucine (referred to as F537_K539>L mutation), and substitution of lysine 539 with leucine (referred to as K539L (TT) mutation or K539L (CT) mutation). As a result of the K539L (TT) mutation, a codon encoding lysine 539 (AAA) is mutated into a codon encoding leucine (TTA). As a result of the K539L (CT) mutation, a codon encoding lysine 539 (AAA) is mutated into a codon encoding leucine (CTA).

SEQ ID NO: 12 shows a nucleotide sequence encoding exon 12 of the wild-type JAK2 gene. In the presence of the N542_E543del mutation, 6 nucleotides at positions 250 to 255 are deleted from the nucleotide sequence represented by SEQ ID NO: 12. In the presence of the E543_D544del mutation, 6 nucleotides at positions 253 to 258 are deleted from the nucleotide sequence represented by SEQ ID NO: 12. In the presence of the R541_E543>K mutation, 6 nucleotides at positions 248 to 253 are deleted from the nucleotide sequence represented by SEQ ID NO: 12. In the presence of the F537_K539>L mutation, 6 nucleotides at positions 237 to 242 are deleted from the nucleotide sequence represented by SEQ ID NO: 12. In the presence of the K539L (TT) mutation, AA at positions 241 and 242 are substituted with TT in the nucleotide sequence represented by SEQ ID NO: 12. In the presence of the K539L (CT) mutation, AA at positions 241 and 242 are substituted with CT in the nucleotide sequence represented by SEQ ID NO: 12.

### <Kit for Evaluating a Gene Mutation>

The kit for evaluating a gene mutation according to the present invention may comprise the CALR mutation probe described in the <CALR gene mutation>section above, the JAK2 mutation probe described in the <JAK2 gene mutation>section above, or both the CALR mutation probe and the JAK2 mutation probe. The kit for evaluating a gene mutation according to the present invention may simultaneously identify the gene mutations in CALR and/or JAK2 and the gene mutation in MPL. Such gene mutations in CALR, JAK2, and MPL are used for diagnosis of myeloproliferative neoplasms based on the criteria provided by World Health Organization (WHO) (e.g., the 2016 version).

Examples of gene mutations related to myeloproliferative neoplasms in MPL include the W515K mutation (substitution of tryptophan 515 with lysine) and the W515L mutation (substitution of tryptophan 515 with leucine). Such MPL gene mutation is observed in 3% to 5% of patients with essential thrombocythemia (ET) and in 6% to 10% of patients with primary myelofibrosis (PMF). SEQ ID NO: 13 shows a nucleotide sequence encoding wild-type MPL. In the presence of the W515K mutation, TG at positions 305 and 306 are substituted with AA in the nucleotide sequence represented by SEQ ID NO: 13. In the presence of the W515L mutation, G at position 306 is substituted with T in the nucleotide sequence represented by SEQ ID NO: 13.

When the kit for evaluating a gene mutation according to the present invention comprises the CALR mutation probe described in the <CALR gene mutation>section above, any probes for identifying JAK2 and MPL gene mutations can be used. When the kit for evaluating a gene mutation according to the present invention comprises the JAK2 mutation probe described in the <JAK2 gene mutation>section above, any probes for identifying CALR and MPL gene mutations can be used.

Concerning the JAK2 V617F mutation, a more specific example of a mutation probe that can be used is an oligonucleotide comprising CTCCACAGAaACATACTCC (SEQ ID NO: 14) corresponding to the substitution in SEQ ID NO: 11. In the above sequence, a lowercase letter “a” corresponds to substitution of G with T at position 351 in the nucleotide sequence represented by SEQ ID NO: 11. The JAK2 V617F mutation can be identified using a wild-type probe corresponding to wild-type JAK2 (a lowercase letter “a” in the above sequence is substituted with “c”). Specifically, the JAK2 V617F mutation may be identified using a mutation probe comprising the nucleotide sequence represented by SEQ ID NO: 14 or a set of probes comprising the mutation probe and a wild-type probe.

Concerning the JAK2 N542_E543del mutation, an oligonucleotide comprising CACAAAATCAGA-GATTTGATATTTG (SEQ ID NO: 15) can be used as a mutation probe. In the above sequence, a position indicated by a hyphen corresponds to deletion of 6 nucleotides at positions 250 to 255 from the nucleotide sequence represented by SEQ ID NO: 12. Concerning the JAK2 E543_D544del mutation, an oligonucleotide comprising CACAAAATCAGAAAT-TTGATATTTGT (SEQ ID NO: 16) can be used as a mutation probe. In the above sequence, a position indicated by a hyphen corresponds to deletion of 6 nucleotides at positions 253 to 258 from the nucleotide sequence represented by SEQ ID NO: 12. Concerning the JAK2 R541_E543>K mutation, an oligonucleotide comprising CACAAAATCA-AAGATTTGATATTTGT (SEQ ID NO: 17) can be used as a mutation probe. In the above sequence, a position indicated by a hyphen corresponds to deletion of 6 nucleotides at positions 248 to 253 from the nucleotide sequence represented by SEQ ID NO: 12. Concerning the JAK2 F537_K539>L mutation, an oligonucleotide comprising CCAAATGGTG-TTAATCAGAAATGAA (SEQ ID NO: 18) can be used as a mutation probe. In the above sequence, a position indicated by a hyphen corresponds to deletion of 6 nucleotides at positions 237 to 242 from the nucleotide sequence represented by SEQ ID NO: 12. Concerning the JAK2 K539L (TT) mutation, an oligonucleotide comprising GGTGTTTCACttAATCAGAAATGA (SEQ ID NO: 19) can be used as a mutation probe. In the above sequence, lowercase letters “tt” correspond to AA at positions 241 and 242 in the nucleotide sequence represented by SEQ ID NO: 12. Concerning the JAK2 K539L (CT) mutation, an oligonucleotide comprising GTGTTTCACctAATCAGAAATGA (SEQ ID NO: 20) can be used as a mutation probe. In the above sequence, lowercase letters “ct” correspond to AA at positions 241 and 242 in the nucleotide sequence represented by SEQ ID NO: 12.

Various JAK2 exon 12 mutations can be identified using wild-type probes corresponding to wild-types of relevant mutants. Various mutants described above are very close to each other or partially overlapped with each other. Thus, a representative wild-type probe can be used or several wild-type probes can be used in combination. In the examples described below, a wild-type probe designed to have the N542_E543del mutation site, the E543_D544del mutation site, and the R541_E543>K mutation site at the center thereof and a wild-type probe designed to have the F537_K539>L mutation site at the center thereof were used.

Concerning the CALR type 1 mutation, an example of a probe corresponding to the 52-bp deletion in SEQ ID NO: 10 that can be used is an oligonucleotide comprising CTCCTTGT-CCGCTCCTCGTC (SEQ ID NO: 21). In the above sequence, a position indicated by a hyphen corresponds to deletion of 52 nucleotides at positions 506 to 557 from the nucleotide sequence represented by SEQ ID NO: 10. The CALR type 1 mutation can be identified using a wild-type probe corresponding to wild-type CALR. Specifically, the CALR type 1 mutation may be identified using a mutation probe comprising the nucleotide sequence represented by SEQ ID NO: 21 or a set of probes comprising the mutation probe and a wild-type probe.

Concerning the CALR type 2 mutation, for example, an oligonucleotide comprising ATCCTCCgacaaTTGTCCT (SEQ ID NO: 22) corresponding to the insertion of 5 nucleotides in SEQ ID NO: 10 can be used as a probe. In the above sequence, lowercase letters “gacaa” indicate insertion of 5 nucleotides. The CALR type 2 mutation can be identified using a wild-type probe corresponding to wild-type CALR. Specifically, the CALR type 2 mutation may be identified using a mutation probe comprising the nucleotide sequence represented by SEQ ID NO: 22 or a set of probes comprising the mutation probe and a wild-type probe.

Concerning the MPL W515K mutation, for example, an oligonucleotide comprising GAAACTGCttCCTCAGCA (SEQ ID NO: 23) corresponding to the substitution in SEQ ID NO: 13 can be used as a mutation probe. In the above sequence, lowercase letters “tt” correspond to substitution of TG at positions 305 and 306 with AA in the nucleotide sequence represented by SEQ ID NO: 13. The MPL W515K mutation can be identified using a wild-type probe (lowercase letters “tt” are substituted with “ca” in the above sequence) corresponding to wild-type MPL. Specifically, the MPL W515K mutation may be identified using a mutation probe comprising the nucleotide sequence represented by SEQ ID NO: 23 or a set of probes comprising the mutation probe and a wild-type probe.

Concerning the MPL W515L mutation, for example, an oligonucleotide comprising GGAAACTGCAaCCTCAG (SEQ ID NO: 24) corresponding to the substitution in SEQ ID NO: 13 can be used as a mutation probe. In the above sequence, a lowercase letter “a” corresponds to substitution of G with T at position 306 in the nucleotide sequence represented by SEQ ID NO: 13. The MPL W515L mutation can be identified using a wild-type probe corresponding to wild-type MPL (a lowercase letter “a” in the above sequence is substituted with “c”). Specifically, the MPL W515L may be identified using a mutation probe comprising the nucleotide sequence represented by SEQ ID NO: 24 or a set of probes comprising the mutation probe and a wild-type probe.

As CALR mutation probes having mismatches used to identify the type 1 mutation, the type 3 mutation, the type 4 mutation, and/or the type 5 mutation in CALR, nucleotide sequences represented by SEQ ID NOs: 95, 53, 54, and 55 were exemplified above. The nucleotide sequences of the CALR mutation probes are not limited to those represented by SEQ ID NOs: 95, 53, 54, and 55 and can be adequately designed based on the nucleotide sequence of the type 1 mutation represented by SEQ ID NO: 57, the nucleotide sequence of the type 3 mutation represented by SEQ ID NO: 58, the nucleotide sequence of the type 4 mutation represented by SEQ ID NO: 59, and the nucleotide sequence of the type 5 mutation represented by SEQ ID NO: 60.

Also, mutation probes used to identify gene mutations in JAK2 were exemplified. The nucleotide sequences of the mutation probes are not limited to those represented by SEQ ID NOs: 14 to 20 and can be adequately designed based on the nucleotide sequences of JAK2 represented by SEQ ID NOs: 11 and 12. As mutation probes used to identify the type 1 mutation and the type 2 mutation in CALR, nucleotide sequences represented by SEQ ID NOs: 21 and 22 were exemplified above. The nucleotide sequences of the mutation probes are not limited to those represented by SEQ ID NOs: 21 and 22 and can be adequately designed based on the nucleotide sequence of CALR represented by SEQ ID NO: 10. While nucleotide sequences represented by SEQ ID NOs: 23 and 24 were exemplified as probes used to identify gene mutations in MPL, the nucleotide sequences of the mutation probes are not limited to those represented by SEQ ID NOs: 23 and 24 and can be adequately designed based on the nucleotide sequence of MPL represented by SEQ ID NO: 13.

The length of a probe sequence is not particularly limited. For example, a sequence can comprise 10 to 30 nucleotides, and a sequence preferably comprises 15 to 25 nucleotides. As described above, a probe can comprise, for example 10 to 30 nucleotides, and it preferably comprises 15 to 25 nucleotides, which is a total of a nucleotide sequence designed based on a region including a gene mutation in the nucleotide sequences represented by SEQ ID NOs: 10 to 13 and a nucleotide sequence (or nucleotide sequences) added to either or both of the terminuses of the former nucleotide sequence.

The probes designed as described above are preferably nucleic acid probes and more preferably DNA probes. While “DNA” encompasses both a double-stranded DNA and a single-stranded DNA, a single-stranded DNA is preferable. Probes can be obtained via, for example, chemical synthesis using a nucleic acid synthesizer. Examples of nucleic acid synthesizers that can be used include apparatuses referred to as a DNA synthesizer, a fully-automated nucleic acid synthesizer, and a nucleic acid autosynthesizer.

The probes designed as described above are preferably fixed at the 5′ terminuses thereof on the support and used in the form of microarrays (e.g., DNA chips). Microarrays preferably comprise mutation probes and wild-type probes concerning the gene mutations described above. With the use of mutation probes and wild-type probes, a percentage of mutations can be accurately determined, as well as the presence or absence of mutations. A difference in the length of the nucleotide sequence between a mutation probe and a wild-type probe is preferably up to 2 nucleotides, and they are more preferably of the same length.

The microarrays according to the present invention can be prepared by fixing the probes on the support.

A support can be prepared from any material known in the art without particular limitation. Examples of materials include: electrically conductive materials, such as noble metals including platinum, platinum black, gold, palladium, rhodium, silver, mercury, tungsten, and a compound of any thereof and carbon represented by graphite and carbon fibers; silicon materials represented by monocrystalline silicon, amorphous silicon, silicon carbide, silicon oxide, and silicon nitride and composite silicon materials represented by SOI (Silicon on Insulator); inorganic materials, such as glass, quartz glass, alumina, sapphire, ceramics, forsterite, and photosensitive glass; and organic materials, such as polyethylene, ethylene, propropylene, cyclic polyolefin, polyisobutyrene, polyethylene terephthalate, unsaturated polyester, fluorocarbon-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate, polyamide, phenolic resin, urea resin, epoxy resin, melamine resin, styrene-acrylonitrile copolymer, acrylonitrile-butadiene-styrene copolymer, polyphenylene oxide, and polysulfone. While a configuration of a support is not particularly limited, a planar support is preferable.

In the present invention, a support preferably comprises, on its surface, a carbon layer and a chemical modification group. A support comprising a carbon layer and a chemical modification group on its surface encompasses a support comprising a carbon layer and a chemical modification group on a substrate surface and a support comprising a substrate made of a carbon layer and a chemical modification group on the substrate surface. Any substrate material that is known in the art can be used without particular limitation. Materials similar to those exemplified as the support materials can be used.

The microarrays according to the present invention preferably uses a support having a planar microstructure. A configuration of a support may be, but is not limited to, rectangular, square, or round. A support is generally of 1 to 75-mm square, preferably of 1 to 10 mm-square, and more preferably of 3 to 5 mm-square. Since a support with a planer microstructure is easy to produce, use of a substrate of a silicon material or a resin material is preferable, and a support comprising a monocrystalline silicon support and a carbon layer and a chemical modification group on the surface of the silicon substrate is more preferable. Some monocrystalline silicons may suffer from slightly varied orientations of the crystalline axis (may be referred to as “mosaic crystalline”) or disorientation at the atomic scale (lattice defects).

In the present invention, a carbon layer to be provided on a substrate is not particularly limited. Use of synthesized diamond, high-pressure synthesized diamond, natural diamond, soft diamond (e.g., diamond-like carbon), amorphous carbon, any carbon-based material such as graphite, fullerene, or carbon nanotube, a mixture of any thereof, or a laminate of any thereof is preferable. Carbides, such as hafnium carbide, niobium carbide, silicon carbide, tantalum carbide, thorium carbide, titanium carbide, uranium carbide, tungsten carbide, zirconium carbide, molybdenum carbide, chromium carbide, or vanadium carbide, may also be used. The term “soft diamond” used herein generally refers to an imperfect diamond structure, which is a diamond-carbon mixture, such as a so-called diamond-like carbon (DLC), and a mixing ratio is not particularly limited. A carbon layer is advantageous in the following respect. That is, a carbon layer is excellent in chemical stability and it is thus tolerant to subsequent reactions, such as introduction of a chemical modification group or binding to an analyte substance; a carbon layer binds to an analyte substance via electrostatic binding and such binding is thus flexible; a carbon layer is UV-transparent at the time of detection because of a lack of UV absorption; and a carbon layer is electrically conductive at the time of electroblotting. When a carbon layer binds to an analyte substance, in addition, an extent of nonspecific adsorption is low, and it is thus advantageous in that respect. As described above, a support comprising a carbon layer as the substrate may be used.

In the present invention, a carbon layer can be formed in accordance with a conventional technique. Examples include the microwave plasma chemical vapor deposition (CVD) method, the electric cyclotron resonance chemical vapor deposition (ECRCVD) method, the inductive coupled plasma (ICP) method, the direct current sputtering method, the electric cyclotron resonance (ECR) sputtering method, the ionized vapor deposition method, the arc vapor deposition method, the laser vapor deposition method, the electron beam (EB) vapor deposition method, and the resistance heating vapor deposition method.

According to the high-frequency plasma CVD method, a starting material gas (methane) is degraded by a glow discharge caused between electrodes by a high frequency and a carbon layer is synthesized on a substrate. According to the ionized vapor deposition method, thermal electrons formed of tungsten filaments are used to degrade and ionize a starting material gas (benzene) and a carbon layer is formed on a substrate by a bias voltage. In a gas mixture comprising 1% to 99% by volume of hydrogen gas with the balance consisting of 99% to 1% by volume of methane gas, a carbon layer may be formed by the ionized vapor deposition method.

According to the arc vapor deposition method, a direct current is applied to a space between a solid graphite material (the cathodic evaporation source) and a vacuum container (the anode) to cause an arc discharge in vacuum, a carbon atom plasma is generated from the cathode, a bias voltage that is further negative than the evaporation source is applied to a substrate, and carbon ions in the plasma are accelerated toward the substrate. Thus, a carbon layer can be formed.

According to the laser vapor deposition method, for example, a Nd:YAG laser (pulse oscillator) beam is applied to a graphite target substrate to melt the substrate, and carbon atoms are then deposited on the glass substrate. Thus, a carbon layer can be formed.

When a carbon layer is to be formed on a substrate surface, carbon layer thickness is generally of monolayer thickness to approximately 100 μm. When a carbon layer is excessively thin, a surface of the underlayer substrate may be partially exposed. When a carbon layer is excessively thick, in contrast, productivity is deteriorated. Thus, carbon layer thickness is preferably 2 nm to 1 μm, and more preferably 5 nm to 500 nm.

A chemical modification group is introduced onto a substrate surface comprising a carbon layer formed thereon. Thus, oligonucleotide probes can be firmly fixed on the support. A person skilled in the art can select an adequate chemical modification group to be introduced. Examples thereof include, but are not particularly limited to, amino, carboxyl, epoxy, formyl, hydroxyl, and active ester groups.

An amino group can be introduced by, for example, irradiating a carbon layer with ultraviolet rays in ammonia gas or by plasma treatment. Alternatively, an amino group can be introduced by irradiating a carbon layer with ultraviolet rays in chlorine gas to chlorinate the carbon layer and irradiating the chlorinated carbon layer with ultraviolet rays in ammonia gas. Alternatively, an amino group can also be introduced by performing a reaction with a chlorinated carbon layer in a polyvalent amine gas such as methylene diamine or ethylene diamine.

A carboxyl group can be introduced by, for example, allowing an appropriate compound to react with the above-aminated carbon layer. Examples of a compound to be used for introduction of a carboxyl group include: halo carboxylic acid represented by the formula: X—R¹—COOH (wherein X denotes a halogen atom and R¹ denotes a C10-12 divalent hydrocarbon group), such as chloroacetic acid, fluoroacetic acid, bromoacetic acid, iodoacetic acid, 2-chloropropionic acid, 3-chloropropionic acid, 3-chloroacrylic acid, and 4-chlorobenzoic acid; dicarboxylic acid represented by the formula: HOOC—R²—COOH (wherein R² denotes a single bond or C1-12 divalent hydrocarbon group), such as oxalic acid, malonic acid, succinic acid, maleic acid, fumaric acid, and phthalic acid; polyvalent carboxylic acid such as polyacrylic acid, polymethacrylic acid, trimellitic acid, and butane tetracarboxylic acid; keto acid or aldehyde acid represented by the formula: R³—CO—R⁴—COOH (wherein R³ denotes a hydrogen atom or C1-12 divalent hydrocarbon group and R⁴ denotes a C1-12 divalent hydrocarbon group); monohalides of dicarboxylic acid represented by the formula: X—OC—R⁵—COOH (wherein X denotes a halogen atom and R⁵ denotes a single bond or C1-12 divalent hydrocarbon group), such as succinic acid monochloride and malonic acid monochloride; and acid anhydrides such as anhydrous phthalic acid, anhydrous succinic acid, anhydrous oxalic acid, anhydrous maleic acid, and anhydrous butane tetracarboxylic acid.

An epoxy group can be introduced by, for example, allowing an appropriate polyvalent epoxy compound to react with the above-aminated carbon layer. Alternatively, an epoxy group can be introduced by allowing organic peracid to react with a carbon=carbon double bond contained in a carbon layer. Examples of organic peracid include peracetic acid, perbenzoic acid, diperoxyphthalic acid, performic acid, and trifluoro peracetic acid.

A formyl group can be introduced by, for example, allowing glutaraldehyde to react with the above-aminated carbon layer.

A hydroxyl group can be introduced by, for example, allowing water to react with the above-chlorinated carbon layer.

The term “active ester group” refers to an ester group having an electron-withdrawing group with high acidity on the alcohol side of an ester group and activating nucleophilic reaction. Such active ester group specifically refers to an ester group with high reaction activity. An active ester group has an electron-withdrawing group on the alcohol side of the ester group, which is activated to a degree higher than alkyl ester. Such active ester group has reactivity to a group, such as an amino group, a thiol group, or a hydroxyl group. More specifically, phenol esters, thiophenol esters, N-hydroxyamine esters, cyanomethyl esters, esters of heterocyclic hydroxy compounds, and the like are known as active ester groups having activity much higher than that of alkyl esters and the like. More specifically, examples of such active ester groups include a p-nitro phenyl group, an N-hydroxysuccinimide group, a succinimide group, a phthalic imide group, and a 5-norbornene-2,3-dicarboxyimide group. In particular, an N-hydroxysuccinimide group is preferably used.

An active ester group can be introduced by performing active-esterification of the above-introduced carboxyl group using a dehydrating and condensing agent, such as cyanamide and carbodiimide (e.g., 1-[3-(dimethylamino)propyl]-3-ethyl carbodiimide), and a compound, such as N-hydroxysuccinimide. As a result of this treatment, a group in which an active ester group such as an N-hydroxysuccinimide group is bound to the terminus of a hydrocarbon group via amide bond can be formed (JP 2001-139532 A).

Probes are dissolved in a spotting buffer to prepare a spotting solution, the resulting spotting solution is fractionated to each well of a 96-well or 384-well plastic plate, the fractionated solutions are spotted on a support using a spotter apparatus or the like, and microarrays comprising probes fixed on the support can be thus prepared. Alternatively, a spotting solution may be manually spotted using a micropipetter.

After spotting, it is preferable to perform incubation, so as to proceed a binding reaction of probes to a support. Incubation is generally performed at −20° C. to 100° C., and preferably 0° C. to 90° C., generally for 0.5 to 16 hours, and preferably for 1 to 2 hours. It is desirable to perform incubation at high humidity, such as 50% to 90% humidity. Following incubation, it is preferable to wash the support using a wash solution (e.g., 50 mM TBS/0.05% Tween 20, 2×SSC/0.2% SDS solution, or ultrapure water) to remove DNAs that have not bound to the support.

With the use of the microarrays constituted as described above, the presence or absence of the gene mutations in JAK2, CALR, and MPL of a target of diagnosis can be simultaneously determined.

Specifically, the presence or absence of the gene mutations in JAK2, CALR, and MPL is determined by a method comprising: a step of extracting DNA from a sample obtained from a target of diagnosis; a step of amplifying regions including the gene mutation in JAK2 (i.e., a region including the V617F mutation and a region including a gene mutation in exon 12 of JAK2), a region including the gene mutation in CALR, and a region including the gene mutation in MPL with the use of the extracted DNA as a template; and a step of detecting the presence or absence of the gene mutations in JAK2, CALR, and MPL included in the amplified nucleic acids with the use of the microarray described above.

A target of diagnosis is generally a human. While a human race is not particularly limited, a target of diagnosis is a person of the yellow race, preferably a person of East Asian ethnicity, and more preferably a person of Japanese ethnicity. A target of diagnosis can be a patient suspected of myeloproliferative neoplasms.

A sample obtained from a target of diagnosis is not particularly limited. Examples include blood-related samples, such as blood, serum, and plasma samples, lymph fluid, feces, cancer cells, and fractured and extracted tissue or organs.

At the outset, DNA is extracted from a sample obtained from a target of diagnosis. A method of extraction is not particularly limited. Examples of methods that can be employed include DNA extraction methods involving the use of phenol-chloroform, ethanol, sodium hydroxide, and CTAB.

Subsequently, an amplification reaction is performed using the obtained DNA as a template to amplify regions including JAK2 (a region including the V617F mutation and a region including a gene mutation in exon 12 of JAK2), a region including CALR, and a region including MPL. Examples of amplification reactions that can be adopted include the polymerase chain reaction (PCR) method, the loop-mediated isothermal amplification (LAMP) method, and the isothermal and chimeric primer-initiated amplification of nucleic acids (ICAN) method. In an amplification reaction, a label is preferably added, so that the amplified region can be identified. In such a case, a method of labeling an amplified nucleic acid is not particularly limited. For example, primers used for the amplification reaction may be labeled in advance, or a labeled nucleotide may be used as a substrate for the amplification reaction. While label substances are not particularly limited, a radioactive isotope, a fluorescent dye, an organic compound such as digoxigenin (DIG) or biotin, or the like can be used.

The reaction system comprises, for example, a buffer necessary for nucleic acid amplification and labeling, heat-tolerant DNA polymerase, primers specific to the regions to be amplified, labeled nucleotide triphosphate (specifically, fluorescence-labeled nucleotide triphosphate), nucleotide triphosphate, and magnesium chloride.

When the kit for evaluating a gene mutation according to the present invention comprises the CALR mutation probe described in the “CALR gene mutation” section above, the kit can comprise a set of primers that amplifies regions including the type 1 mutation, the type 3 mutation, the type 4 mutation, and the type 5 mutation in CALR; that is, regions including the site of deletion (indicated by arrows in FIG. 1). A set of primers is composed of a pair of a forward primer and a reverse primer.

A region including the site of deletion amplified with the use of a set of primers is detected using the CALR mutation probes having the mismatches (e.g., SEQ ID NOs: 95, 53, 54, and 55). As shown in FIG. 1, the type 1 mutation, the type 3 mutation, the type 4 mutation, and the type 5 mutation each comprise the sequence (underlined in FIG. 1) that is the same as the wild-type on the 3′ side of the site of deletion. Accordingly, probes that are designed to be completely consistent with the type 1 mutation, the type 3 mutation, the type 4 mutation, and the type 5 mutation, including the site of deletion may non-specifically hybridize to wild-type samples.

With the use of the CALR mutation probes (e.g., SEQ ID NOs: 95, 53, 54, and 55), in contrast, a possibility of non-specific hybridization to wild-type samples may be reduced because of the mismatches. With the use of the kit for evaluating a gene mutation according to the present invention, accordingly, samples having the type 1 mutation can be precisely detected separately from wild-type samples. With the use of the kit for evaluating a gene mutation according to the present invention, also, samples having the type 3 mutation can be precisely detected separately from wild-type samples. With the use of the kit for evaluating a gene mutation according to the present invention, also, samples having the type 4 mutation can be precisely detected separately from wild-type samples. With the use of the kit for evaluating a gene mutation according to the present invention, in addition, samples having the type 5 mutation can be precisely detected separately from wild-type samples.

While the design of the CALR mutation probes having mismatches to detect the type 1 mutation, the type 3 mutation, the type 4 mutation, and the type 5 mutation in CALR was described above, mutation probes for detecting mutations other than the CALR gene can be designed in the same manner. When the length of a deletion mutation exceeds a given length and a sequence after the deletion mutation is similar to a wild-type sequence, for example, a mutation probe can be designed in the same manner. In the case of deletion of 5 or more nucleotides, and preferably 10 or more nucleotides, which is longer than a preferable nucleotide length as a probe, and in the presence of a sequence that is consistent with the sequence after deletion by 2 or more nucleotides at positions within 10 nucleotides from the site of deletion in the wild-type nucleotide sequence, more specifically, a mutation probe can be designed to have mismatches in a part of the consistent sequence.

Since the 5′ side of the site of deletion in the wild-type nucleotide sequence is consistent with the sequence after deletion, a CALR mutation probe having mismatches for detecting the type 1 mutation, the type 3 mutation, the type 4 mutation, or the type 5 mutation in CALR having mismatches on the 3′ side of the site of deletion was designed. In the case that the 3′ side of the site of deletion in the wild-type nucleotide sequence is consistent with the sequence after deletion, in contrast, a mutation probe can be designed to have mismatches on the 5′ side of the site of deletion.

When the kit for evaluating a gene mutation according to the present invention comprises the JAK2 mutation probe described in the “JAK2 gene mutation” section above, the kit comprises sets of primers that amplify regions including gene mutations in JAK2; i.e., the set of primers for the V617F mutation that amplifies a region including the V617F mutation and the set of primers for exon 12 that amplifies a region including the gene mutation in exon 12 of the JAK2 gene. A set of primers is composed of a pair of a forward primer and a reverse primer.

The set of primers for the V617F mutation is not particularly limited, provided that a region encoding an amino acid corresponding to valine 617 in the wild-type sequence can be specifically amplified. A person skilled in the art can adequately design such set of primers. An example of a set of primers comprises the forward primer JAK2-F: 5′-GAGCAAGCTTTCTCACAAGCATTTGG-3′ (SEQ ID NO: 25) and the reverse primer JAK2-R: 5′-CTGACACCTAGCTGTGATCCTGAAACTG-3′ (SEQ ID NO: 26).

When amplifying a region including the V617F mutation with the use of the set of primers for the V617F mutation, the concentration of either one of the set of primers for the V617F mutation, for example, a fluorescence-labeled primer (e.g., a forward primer), is preferably adjusted to 1.0 μM or higher. By adjusting the primer concentration to such range, a region including the V617F mutation and a region including the gene mutation in exon 12 can be sufficiently amplified. The upper limit of the primer concentration is not particularly limited, and the upper limit of the primer concentration used in a general nucleic acid amplification reaction can be employed (e.g., 10 μM).

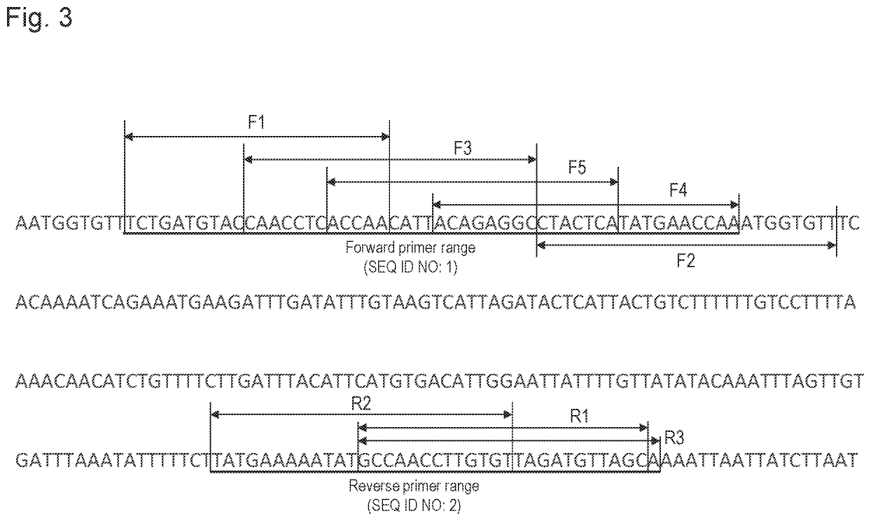

The set of primers for exon 12 is designed to collectively amplify at least 2, preferably 3, more preferably 4, further preferably 5, and most preferably 6 of a plurality of gene mutations included in exon 12. More specifically, the set of primers for exon 12 can be composed of the forward primer for exon 12 comprising 10 or more continuous nucleotides selected from the nucleotide sequence represented by SEQ ID NO: 1 and the reverse primer for exon 12 comprising 10 or more continuous nucleotides selected from the nucleotide sequence represented by SEQ ID NO: 2, as shown in FIG. 3. SEQ ID NO: 1 corresponds to positions 178 to 228 of the nucleotide sequence encoding exon 12 and SEQ ID NO: 2 corresponds to positions 399 to 435 thereof. SEQ ID NOs: 1 and 2 are both a partical sequence of SEQ ID NO: 12. All of the 6 gene mutations described above are located between SEQ ID NOs: 1 and 2.

More specifically, the forward primer for exon 12 can be a primer selected from the group consisting of the forward primer F1 for exon 12 comprising the nucleotide sequence represented by SEQ ID NO: 3, the forward primer F3 for exon 12 comprising the nucleotide sequence represented by SEQ ID NO: 4, the forward primer F4 for exon 12 comprising the nucleotide sequence represented by SEQ ID NO: 5, and the forward primer F5 for exon 12 comprising the nucleotide sequence represented by SEQ ID NO: 6.

Specifically, the reverse primer for exon 12 can be a primer selected from the group consisting of the reverse primer R1 for exon 12 comprising the nucleotide sequence represented by SEQ ID NO: 7, the reverse primer R2 for exon 12 comprising the nucleotide sequence represented by SEQ ID NO: 8, and the reverse primer R3 for exon 12 comprising the nucleotide sequence represented by SEQ ID NO: 9.

It is particularly preferable that the set of primers for exon 12 be composed of the forward primer F5 for exon 12 comprising the nucleotide sequence represented by SEQ ID NO: 6 and the reverse primer R2 for exon 12 comprising the nucleotide sequence represented by SEQ ID NO: 8.

The nucleotide sequences of the forward primers F1 and F3 to F5 and the reverse primers R1 to R3 are represented with reference to the corresponding positions in the nucleotide sequence encoding exon 12. Accordingly, either of the forward and reverse primers constituting a set of primers is a complementary strand of the nucleotide sequence represented by a relevant sequence identification number. In the examples below, all the reverse primers are complementary strands.

When amplifying a region including a gene mutation in exon 12 with the use of the set of primers for exon 12, the concentration of either one of the set of primers for exon 12, for example, a fluorescence-labeled primer (e.g., a reverse primer), is preferably adjusted to 2.5 μM or higher. By adjusting the primer concentration to such range, a region including the V617F mutation and a region including the gene mutation in exon 12 can be sufficiently amplified. The upper limit of the primer concentration is not particularly limited, and the upper limit of the primer concentration used in a general nucleic acid amplification reaction can be employed (e.g., 10 μM).

The concentration of the forward primer and that of the reverse primer in a set of primers may be the same with or different from each other, and such conditions are not limited to the set of primers for exon 12. When concentrations are different from each other, it is sufficient if either of the primers satisfies the conditions. In the examples below, the concentration of a fluorescence-labeled primer is set higher in either sets of primers for JAK2 V617F, exon 12, CALR, and MPL.

It is preferable that the ratio of the concentration of a labeled primer in the set of primers for the V617F mutation to the concentration of a labeled primer in the set of primers for exon 12 [the concentration of the primer for exon 12]/[the concentration of the primer for the V617F mutation] be adjusted to 1.0 to 5.5. By adjusting the concertation ratio within such range, a region including the V617F mutation and a region including the gene mutation in exon 12 can be sufficiently amplified.

Primers used for an amplification reaction of a region including the gene mutation in CALR are not particularly limited, provided that the region including the gene mutation can be specifically amplified. A person skilled in the art can adequately design such primers. An example of a set of primers comprises the primer CALR-F: 5′-CGTAACAAAGGTGAGGCCTGGT-3′ (SEQ ID NO: 27) and the primer CALR-R: 5′-GGCCTCTCTACAGCTCGTCCTTG-3′ (SEQ ID NO: 28).

Primers used for an amplification reaction of a region including the gene mutation in MPL are not particularly limited, provided that the region including the gene mutation can be specifically amplified. A person skilled in the art can adequately design such primers. An example of a set of primers comprises the primer MPL-F: 5′-CTCCTAGCCTGGATCTCCTTGG-3′ (SEQ ID NO: 29) and the primer MPL-R: 5′-ACAGAGCGAACCAAGAATGCCTGTTTAC-3′ (SEQ ID NO: 30).

A nucleic acid fragment to be amplified by primers is not particularly limited, provided that such fragment includes a region corresponding to the designed probe. For example, a length of such fragment is preferably 1 kbp or shorter, more preferably 800 bp or shorter, further preferably 500 bp or shorter, and particularly preferably 350 bp or shorter.

The amplified nucleic acids thus obtained are subjected to hybridization to probes fixed on a support, and hybridization between the amplified nucleic acids and the mutation probes is detected. Thus, the presence or absence of the gene mutation in a target of diagnosis can be evaluated. Specifically, hybridization of the amplified nucleic acids to the mutation probes can be assayed by detecting, for example, labels.

When a fluorescent label is used, for example, the fluorescence signal from the label can be detected using a fluorescence scanner, the detected signal is analyzed using image analysis software, and the signal intensity can be thus digitized. Hybridization is preferably carried out under stringent conditions. Under stringent conditions, a specific hybrid is formed, but a non-specific hybrid is not formed. For example, hybridization at 50° C. for 16 hours is followed by washing in the presence of 2×SSC/0.2% SDS at 25° C. for 10 minutes and in the presence of 2×SSC at 25° C. for 5 minutes. Hybridization can be carried out at 45° C. to 60° C. and salt concentration of 0.5×SSC. When a probe chain length is short, hybridization is preferably carried out at temperature lower than the temperature indicated above. When a probe chain length is long, in contrast, hybridization is preferably carried out at temperature higher than the temperature indicated above. At higher salt concentration, hybridization temperature with specificity is increased. At lower salt concentration, in contrast, hybridization temperature with specificity is decreased.

When microarrays comprising mutation probes and wild-type probes are used to detect the gene mutations described above, signal intensities from such mutation probes and wild-type probes can be used to evaluate the presence or absence of the gene mutations. Specifically, the signal intensities from the wild-type probes and the signal intensities from the mutation probes are each measured to determine the judgement value for evaluation of signal intensities derived from the mutation probes. For example, the judgement value can be determined in accordance with the formula: [Signal intensity from mutation probe]/[Signal intensity from wild-type probe]+[Signal intensity from mutation probe].

The judgement value determined in accordance with the formula above is compared with the threshold defined in advance (the cut-off value). When the judgement value is higher than the threshold, the gene mutation described above is determined to be included in the amplified nucleic acid. When the judgement value is lower than the threshold, the gene mutation described above is determined not to be included in the amplified nucleic acid. With the use of the judgement value, accordingly, the presence or absence of the gene mutations in JAK2, CALR, and MPL can be determined.

While the threshold is not particularly limited, the threshold can be defined based on the judgment value determined in accordance with the above formula using a sample, which has been verified to comprise, for example, wild-type gene mutations in JAK2, CALR, and MPL. More specifically, a plurality of samples, which have been verified to comprise wild-type gene mutations in JAK2, CALR, and MPL, can be used to determine a plurality of judgement values, and a total of the mean of such a plurality of judgement values and 3σ (σ: the standard error) can be determined to be the threshold. Alternatively, a total of the average and 2σ or the average and 6 can be used as the threshold.

With the use of the microarrays comprising mutation probes for identification of the gene mutations in JAK2, CALR, and MPL, as described above, the gene mutations in JAK2, CALR, and MPL can be simultaneously identified. Information concerning the gene mutations in JAK2, CALR, and MPL can be used for diagnosis of myeloproliferative neoplasms in accordance with, for example, the criteria defined by WHO (the 2016 version). According to the criteria defined by WHO, specifically, a requirement for diagnosis of polycythemia vera (PV) is the presence of the gene mutation in JAK2. According to the criteria defined by WHO, also, a requirement for diagnosis of essential thrombocythemia (ET) is the presence of the gene mutation in JAK2, CALR, and MPL. According to the criteria defined by WHO, in addition, a requirement for diagnosis of prefibrotic/early primary myelofibrosis (prefibrotic/early PMF) or primary myelofibrosis (PMF) is the presence of the gene mutation in JAK2, CALR, or MPL.

As described above, diagnosis of myeloproliferative neoplasms with the use of, for example, the criteria defined by WHO (the 2016 version) can be performed with the use of microarrays comprising mutation probes for identification of the gene mutations in JAK2, CALR, and MPL.

### EXAMPLES

Hereafter, the present invention is described in greater detail with reference to the examples, although the technical scope of the present invention is not limited to the examples.

### Example 1

### 1. Sample Preparation

In this example, genome DNA derived from the peripheral blood of a healthy individual (Biochain) was used as a wild-type sample.

In order to detect the gene mutations shown in Table 1, target regions (4 regions) including such gene mutations were amplified in this example.

TABLE 1

| | Gene mutation | | Other description |
|---|---|---|---|
| Target region | (Amino acid) | Gene mutation (CDS) | for the mutation |
| JAK2 exon 14 | V617F | c.1849G > T | |
| MPL | W515L | c.1544G > T | |
| | W515K | c.1543_1544TG > AA | |
| CALR | L367fs*46 | c.1092_1143del52 | Type 1 |
| | K385fs*47 | c.1154_1155insTTGTC | Type 2 |
| | L367fs*48 | c.1095_1140del46 | Type 3 |
| | K368fs*51 | c.1102_1135del34 | Type 4 |
| | E364fs*46 | c.1091_1142del52 | Type 5 |
| JAK2 exon 12 | N542_E543del | c.1624_1629delAATGAA | |
| | E543_D544del | c.1627_1632delGAAGAT | |
| | R541_E543 > K | c.1622_1627delGAAATG | |
| | F537_K539 > L | c.1611_1616delTCACAA | |
| | K539L (TT) | c.1615_1616AA > TT | |
| | K539L (CT) | c.1615_1616AA > CT | |

In this example, the primers shown in Table 2 were designed to amplify the 4 target regions shown in Table 1. It should be noted that exon12-F is F5 and exon12-R is a complementary strand of R2.

TABLE 2

| Target region | Name | Sequence (5′-3′) | Labeling |
|---|---|---|---|
| JAK2 V617F | JAK2-F | GAGCAAGCTTTCTCACAAGCATTTGG | IC5-labeled |
| mutation site | JAK2-R | CTGACACCTAGCTGTGATCCTGAAACTG | Not labeled |
| MPL | MPL-F | CTCCTAGCCTGGATCTCCTTGG | IC5-labeled |
| | MPL-R | ACAGAGCGAACCAAGAATGCCTGTTTA | Not labeled |
| | | C | |
| CALR | CALR-F | CGTAACAAAGGTGAGGCCTGGT | IC5-labeled |
| | CALR-R | GGCCTCTCTACAGCTCGTCCTTG | Not labeled |
| JAK exon 12 | exon12-F | ACCAACATTACAGAGGCCTACTCA | Not labeled |
| | exon12-R | ACACAAGGTTGGCATATTTTTCATA | IC5-labeled |

DNA samples prepared in the manner described above were used to amplify the 4 target regions in the JAK2 gene, the CALR gene, and the MPL gene by PCR. PCR was carried out using the template genome DNA at 8 or 16 ng/μ1. The reaction composition is shown in Table 3.

TABLE 3

| Reagent | Manufacturer | Volume (μl) |
|---|---|---|
| 10 × PCR Buffer | Roche Diagnostics | 2.0 |
| 10 nM dNTP mix | Roche Diagnostics | 0.4 |
| Faststart DNA taq polymerase | Roche Diagnostics | 0.2 |
| Primer mix | Life Technologies Japan | 2.0 |
| DNA sample (8 or 16 ng/μl) | | 5.0 |
| Purified water | | 10.4 |

PCR was first carried out at 95° C. for 5 minutes, a cycle of 95° C. for 30 seconds, 59° C. for 30 seconds, and 72° C. for 45 seconds was repeated 40 times, and the subsequent step was carried out at 72° C. for 10 minutes, followed by maintenance at 4° C. in the end.

### 2. Microarrays

In this example, mutation probes corresponding to the V617F mutation and 6 gene mutations in exon 12 of the JAK2 gene, the type 1 mutation to the type 5 mutation in the CALR gene, and the W515L/K mutation in the MPL gene and wild-types probes corresponding to such mutation probes were designed. Table 4 shows the nucleotide sequences of the probes.

TABLE 4

| Probe | Sequence (5′-3′) | SEQ ID NO: |
|---|---|---|
| JAK2 V617 wild-type | TTTTTTTTTTTTCTCCACAGACACATACTCC | 35 |
| JAK2 V617F | TTTTTTTTTTTTCTCCACAGAAACATACTCC | 36 |
| MPL W515 wild-type | TTTTTTTTTTTTAAACTGCCACCTCAGC | 37 |
| MPL W515L | TTTTTTTTTTTTGGAAACTGCAACCTCAG | 38 |
| MPL W515K | TTTTTTTTTTTTGAAACTGCTTCCTCAGCA | 39 |
| CALR wild-type 1 | TTTTTTTTTTTTCTCTTTGCGTTTCTTGTCTTCT | 40 |
| CALR Type 1 | TTTTTTTTTTTTCTCCTTGTCCGCTCCTCGTC | 41 |
| CALR wild-type 2 | TTTTTTTTTTTTCTCATCATCCTTGTCCTCTGC | 42 |
| CALR Type 2 | TTTTTTTTTTTTATCCTCCGACAATTGTCCT | 43 |
| CALR Type 3 | TTTTTTTTTTTTTTGTCCTCTGCTCTGCTCCTC | 61 |
| CALR Type 4 | TTTTTTTTTTTTCTCTGCCTCCTCTAAGCCTCTG | 62 |
| CALR Type 5 | TTTTTTTTTTTTCTCCTTGTCCGCCCCTCGTC | 63 |
| JAK2 exon12 wild-type 1 | TTTTTTTTTTTTTCACAAAATCAGAAATGAAGATTTGATATTTG | 44 |
| JAK2 exon12 N542_E543del | TTTTTTTTTTTTTCACAAAATCAGAGATTTGATATTTG | 45 |
| JAK2 exon12 E543_D544del | TTTTTTTTTTTTTTTTCACAAAATCAGAAATTTGATATTTGT | 46 |
| JAK2 exon12 R541_E543>K | TTTTTTTTTTTTTGTTTCACAAAATCAAAGATTTGATATTTGT | 47 |
| JAK2 exon12 wild-type 2 | TTTTTTTTTTTTTAATGGTGTTTCACAAAATCAGAAAT | 48 |
| JAK2 exon12 F537_1(539>L | TTTTTTTTTTTTTCCAAATGGTGTTAATCAGAAATGAA | 49 |
| JAK2 exon12 K539L (TT) | TTTTTTTTTTTTTTGGTGTTTCACTTAATCAGAAATGA | 50 |
| JAK2 exon12 K539L (CT) | TTTTTTTTTTTTTGTGTTTCACCTAATCAGAAATGA | 51 |

### 3. Identification of Gene Mutation

Hybridization was carried out in the manner described below using a chip comprising the probes described above. At the outset, a moist box was introduced into a chamber set at a designated temperature (52° C.), and the chamber and the moist box were sufficiently preheated. A PCR solution (4 μl) was mixed with 2 μl of a hybridization buffer (2.25×SSC/0.23% SDS/0.2 nM IC5-labeled oligo DNA, Life Technologies Japan), 3 μl of a solution was fractionated from the mixture, the fractionated solution was added dropwise to a convex portion at the center of a hybridization cover, the cover was mounted on the chip, and the reaction was allowed to proceed in the hybridization chamber (Toyo Kohan Co. Ltd.) set at 52° C. for 1 hour. After the completion of the hybridization reaction, the chip from which the hybridization cover had been removed was mounted on a holder, and a stainless-steel washing holder was soaked in a 0.1×SSC/0.1% SDS solution. After the holder was shaken up and down several times, the holder was kept soaked in a 1×SSC solution (room temperature) until the fluorescence intensity of the chip was detected.

Immediately before detection, a cover film was overlaid on the chip, and the fluorescence intensity of the chip was detected using BIOSHOT (Toyo Kohan Co., Ltd.). With the use of the fluorescence intensity derived from the wild-type probe and the fluorescence intensity derived from the mutation probe measured in the manner described above, the judgement values for the JAK2 gene mutations (the V617F mutation and 6 gene mutations in exon 12), the CALR gene mutation, and the MPL gene mutation were determined in accordance with the formula below.

Judgement value=[Fluorescence intensity of mutation probe]/([Fluorescence intensity of wild-type probe]+[Fluorescence intensity of mutation probe])

### Experimentation Example 1-1

In this experimentation example, a plurality of CALR mutation probes used to detect a deletion-type gene mutation in CALR; i.e., the type 3 mutation, the type 4 mutation, or the type 5 mutation, were designed and evaluated. Specifically, a plurality of CALR mutation probes that are completely consistent with the regions including the deletion sites (indicated by arrows in FIG. 1) of the type 3 mutation, the type 4 mutation, and the type 5 mutation and a plurality of CALR mutation probes having mismatches in such regions were designed (Table 5). The probes actually produced each comprise a linker (a region of continuous Ts) bound to the 5′-side of a complementary strand of the designed sequence.

TABLE 5

| Probe | Probe sequence (5′-3′) | Remarks | SEQ ID NO: |
|---|---|---|---|
| Type 3 mutation probe 1 | GAGGAGCAGAGGCAGA | Fully matched | 64 |
| Type 3 mutation probe 2 | GAGCAGAGGCAGAGGA | Fully matched | 65 |
| Type 3 mutation probe 3 | AGGAGCAGAGGCAGAG | Fully matched | 66 |
| Type 3 mutation probe 4 | AGGAGCAGGGCAGAGGA | With mismatches (-A) | 67 |
| Type 3 mutation probe 5 | GAGGAGCAGAGCAGAGGACAA | With mismatches (-G) | 53 |
| Type 3 mutation probe 6 | GAGGAGCAGGCAGAGGACAA | With mismatches (-GA) | 68 |
| Type 3 mutation probe 7 | ACGAGGAGCAGCAGAGGACAA | With mismatches (-GAG) | 69 |
| Type 4 mutation probe 1 | GGCTTAGGAGGAGGCA | Fully matched | 70 |
| Type 4 mutation probe 2 | AGAGGCTTAGGAGGAGG | Fully matched | 71 |
| Type 4 mutation probe 3 | CAGAGGCTTAGGAGGAGG | Fully matched | 72 |
| Type 4 mutation probe 4 | CAGAGGCTTAGAGGAGGCAG | With mismatches (-G) | 73 |
| Type 4 mutation probe 5 | CAGAGGCTTAGAGGAGGCAGAG | With mismatches (-G) | 54 |
| Type 4 mutation probe 6 | GCAGAGGCTTAGAGGAGGCAGA | With mismatches (-G) | 74 |
| Type 4 mutation probe 7 | GCAGAGGCTTAGAGGAGGCA | With mismatches (-G) | 75 |
| Type 4 mutation probe 8 | AGCAGAGGCTTGAGGAGGCAGA | With mismatches (-AG) | 76 |
| Type 4 mutation probe 9 | GCAGAGGCTTGAGGAGGCAGA | With mismatches (-AG) | 77 |
| Type 4 mutation probe 10 | GCAGAGGCTTGAGGAGGCA | With mismatches (-AG) | 78 |
| Type 4 mutation probe 11 | GCAGAGGCTTGGAGGAGGCA | With mismatches (-A) | 79 |
| Type 4 mutation probe 12 | AGCAGAGGCTTGGAGGAGGCA | With mismatches (-A) | 80 |
| Type 4 mutation probe 13 | AGCAGAGGCTTAAGGAGGCAGAG | With mismatches (-GG) | 81 |
| Type 4 mutation probe 14 | AGCAGAGGCTTAAGGAGGCAGA | With mismatches (-GG) | 82 |
| Type 5 mutation probe 1 | GGGGCAGAGGACAAGG | Fully matched | 83 |
| Type 5 mutation probe 2 | GGGCAGAGGACAAGG | Fully matched | 84 |
| Type 5 mutation probe 3 | GACGAGGGGCGGACAAGGA | With mismatches (-AGA) | 85 |
| Type 5 mutation probe 4 | GACGAGGGGCGGACAAGGAG | With mismatches (-AGA) | 55 |
| Type 5 mutation probe 5 | ACGAGGGGCGGACAAGGAG | With mismatches (-AGA) | 86 |
| Type 5 mutation probe 6 | CGAGGGGCGGACAAGGA | With mismatches (-AGA) | 87 |
| Type 5 mutation probe 7 | CGAGGGGCGGACAAGG | With mismatches (-AGA) | 88 |

In this experimentation example, the fluorescence intensity to mutation model samples (100% of mutation plasmids) and the fluorescence intensity to wild-type model samples (plasmids) were measured using the probes shown in Table 5. The results are shown in Table 6. In Table 6, “Specific fluorescence intensity*1” indicates the fluorescence intensity to the mutation model samples and “Non-specific fluorescence intensity*2” indicates the fluorescence intensity to the wild-type model samples.

TABLE 6

| | Probe's | | Specific | Non-specific | |
|---|---|---|---|---|---|
| | nucleotide | Tm | fluorescence | fluorescence | |
| Probe | number | (° C) | intensity*1 | intensity*2 | Remarks |
| Type 3 mutation probe 1 | 16 | 53.6 | 4568 | 193 | Fully matched |
| Type 3 mutation probe 2 | 16 | 53.6 | 4729 | 54 | Fully matched |
| Type 3 mutation probe 3 | 16 | 53.6 | 6223 | 85 | Fully matched |
| Type 3 mutation probe 4 | 17 | 58.5 | 1452 | 60 | With mismatches (-A) |
| Type 3 mutation probe 5 | 21 | 58.7 | 16449 | 128 | With mismatches (-G) |
| Type 3 mutation probe 6 | 20 | 59.2 | 11292 | 4024 | With mismatches (-GA) |
| Type 3 mutation probe 7 | 21 | 60.6 | 10480 | 183 | With mismatches (-GAG) |
| Type 4 mutation probe 1 | 16 | 53.7 | 10891 | 57 | Fully matched |
| Type 4 mutation probe 2 | 17 | 52.5 | 3146 | 506 | Fully matched |
| Type 4 mutation probe 3 | 18 | 53.9 | 11637 | 2512 | Fully matched |
| Type 4 mutation probe 4 | 20 | 56.9 | 12227 | 123 | With mismatches (-G) |
| Type 4 mutation probe 5 | 22 | 58.7 | 25854 | 362 | With mismatches (-G) |
| Type 4 mutation probe 6 | 22 | 60.6 | 28914 | 1700 | With mismatches (-G) |
| Type 4 mutation probe 7 | 20 | 59.0 | 17838 | 418 | With mismatches (-G) |
| Type 4 mutation probe 8 | 22 | 62.5 | 18071 | 771 | With mismatches (-AG) |
| Type 4 mutation probe 9 | 21 | 61.5 | 18896 | 935 | With mismatches (-AG) |
| Type 4 mutation probe 10 | 19 | 60.0 | 5276 | 235 | With mismatches (-AG) |
| Type 4 mutation probe 11 | 20 | 62.2 | 25213 | 2301 | With mismatches (-A) |
| Type 4 mutation probe 12 | 21 | 63.2 | 26570 | 2659 | With mismatches (-A) |
| Type 4 mutation probe 13 | 23 | 60.6 | 21704 | 14782 | With mismatches (-GG) |
| Type 4 mutation probe 14 | 22 | 60.2 | 11730 | 14119 | With mismatches (-GG) |
| Type 5 mutation probe 1 | 16 | 55.5 | 27391 | 3370 | Fully matched |
| Type 5 mutation probe 2 | 15 | 52.2 | 13788 | 4611 | Fully matched |
| Type 5 mutation probe 3 | 19 | 62.1 | 12358 | 57 | With mismatches (-AGA) |
| Type 5 mutation probe 4 | 20 | 62.5 | 26281 | 114 | With mismatches (-AGA) |
| Type 5 mutation probe 5 | 19 | 62.1 | 16923 | 111 | With mismatches (-AGA) |
| Type 5 mutation probe 6 | 17 | 60.2 | 2997 | 12 | With mismatches (-AGA) |
| Type 5 mutation probe 7 | 16 | 59.0 | 1863 | 49 | With mismatches (-AGA) |

As shown in Table 6, a probe having a deletion-type mismatch at a given site was found to be capable of specifically hybridizing to a mutation sample exhibiting high specific fluorescence intensity*1 and low non-specific fluorescence intensity*2. As shown in Table 6, a probe capable of hybridizing specifically to a mutation sample was designed (specific fluorescence intensity*1 of 10,000 or higher and non-specific fluorescence intensity*2 of 1,000 or lower). Among the designed mutation probes, in addition, probes capable of hybridizing to mutation samples with very high specificity were designed (specific fluorescence intensity*1 of 15,000 or higher and non-specific fluorescence intensity*2 of 500 or lower) (e.g., the type 3 mutation probe 5, the type 4 mutation probe 5, the type 4 mutation probe 7, the type 5 mutation probe 4, and the type 5 mutation probe 5). The probes used to identify the type 4 mutation are preferably the type 4 mutation probe 5 and the type 4 mutation probe 7, and the type 4 mutation probe 5 is more preferable because of high specific fluorescence intensity*1. The probes used to identify the type 5 mutation are preferably the type 5 mutation probe 4 and the type 5 mutation probe 5, and the type 5 mutation probe 4 is more preferable because of high specific fluorescence intensity*1.

With the use of the probes shown in Table 5, the fluorescence intensity to mutation model samples (the type 1 mutation model plasmid, the type 2 mutation model plasmid, the type 3 mutation model plasmid, the type 4 mutation model plasmid, and the type 5 mutation model plasmid) and the fluorescence intensity to wild-type model samples (plasmids) were measured. The results are shown in Table 7.

TABLE 7

| Probe | Wild-type | Type 1 | Type 2 | Type 3 | Type 4 | Type 5 |
|---|---|---|---|---|---|---|
| Type 3 mutation probe 1 | 193 | 101 | 162 | 4568 | 866 | 34 |
| Type 3 mutation probe 2 | 54 | 61 | 51 | 4729 | 158 | 42 |
| Type 3 mutation probe 3 | 85 | 68 | 119 | 6223 | 260 | 40 |
| Type 3 mutation probe 4 | 60 | 57 | 49 | 1452 | 137 | 22 |
| Type 3 mutation probe 5 | 128 | 134 | 103 | 16449 | 1342 | 31 |
| Type 3 mutation probe 6 | 4024 | 82 | 3079 | 11292 | 16243 | 99 |
| Type 3 mutation probe 7 | 183 | 605 | 138 | 10480 | 3009 | 388 |
| Type 4 mutation probe 1 | 57 | 75 | 72 | 261 | 10891 | 48 |
| Type 4 mutation probe 2 | 506 | 93 | 498 | 101 | 3146 | 72 |
| Type 4 mutation probe 3 | 2512 | 60 | 2186 | 87 | 11637 | 17 |
| Type 4 mutation probe 4 | 123 | 62 | 170 | 103 | 12227 | 21 |
| Type 4 mutation probe 5 | 362 | 94 | 396 | 54 | 25854 | 48 |
| Type 4 mutation probe 6 | 1700 | 72 | 1244 | 53 | 28914 | 34 |
| Type 4 mutation probe 7 | 418 | 66 | 353 | 46 | 17838 | 47 |
| Type 4 mutation probe 8 | 771 | 83 | 643 | 69 | 18071 | 57 |
| Type 4 mutation probe 9 | 935 | 111 | 792 | 102 | 18896 | 39 |
| Type 4 mutation probe 10 | 235 | 78 | 194 | 207 | 5276 | 48 |
| Type 4 mutation probe 11 | 2301 | 85 | 1570 | 39 | 25213 | 36 |
| Type 4 mutation probe 12 | 2659 | 89 | 2002 | 43 | 26570 | 40 |
| Type 4 mutation probe 13 | 14782 | 89 | 12972 | 45 | 21704 | 46 |
| Type 4 mutation probe 14 | 14119 | 86 | 11828 | 62 | 11730 | 44 |
| Type 5 mutation probe 1 | 3370 | 1141 | 121 | 2512 | 11336 | 27391 |
| Type 5 mutation probe 2 | 4611 | 816 | 169 | 2831 | 8242 | 13788 |
| Type 5 mutation probe 3 | 57 | 300 | 23 | 23 | 35 | 12358 |
| Type 5 mutation probe 4 | 114 | 4700 | 96 | 832 | 1191 | 26281 |
| Type 5 mutation probe 5 | 111 | 1219 | 76 | 332 | 624 | 16923 |
| Type 5 mutation probe 6 | 12 | 70 | 99 | 86 | 46 | 2997 |
| Type 5 mutation probe 7 | 49 | 42 | 31 | 17 | 51 | 1863 |

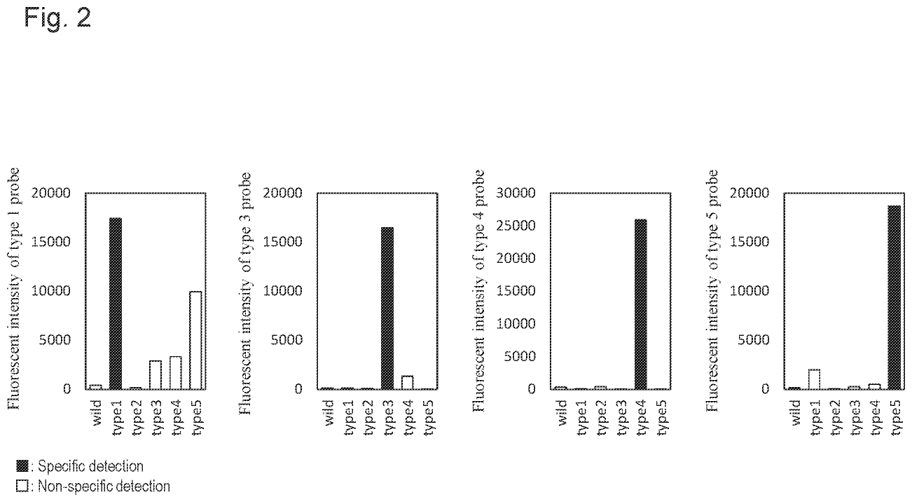

As shown in Table 7, probes each having a deletion-type mismatch at a given site were found to be capable of specifically detecting various mutation types. Of the results shown in Table 7, the results of measurements obtained with the use of the type 3 mutation probe 5, the type 4 mutation probe 5, and the type 5 mutation probe 4 are shown in FIG. 2. Probe sequences of the type 3 mutation probe 5, the type 4 mutation probe 5, and the type 5 mutation probe 4 actually produced are as shown in Table 4. Of the results shown in Experimentation Example 1-2 below, the results of measurements obtained with the use of the type 1 mutation probe 7 (the probe sequence actually prepared is shown in Table 4) are also shown in FIG. 2.

### Experimentation Example 1-2

In this experimentation example, a plurality of CALR mutation probes used to detect a deletion-type gene mutation in CALR; i.e., the type 1 mutation, were designed and evaluated. Specifically, a plurality of CALR mutation probes that are completely consistent with the regions including the deletion sites (indicated by arrows in FIG. 1) of the type 1 mutation and a plurality of CALR mutation probes having mismatches in such regions were designed (Table 8). The probes actually produced each comprise a linker (a region of continuous Ts) bound to the 5′-side of a complementary strand of the designed sequence.

TABLE 8

| Probe | Probe sequence (5′-3′) | Remarks | SEQ ID NO |
|---|---|---|---|
| Mutation type 1 probe 1 | GGAGCAGAGGACAAGGA | Fully matched | 89 |
| Mutation type 1 probe 2 | AGGAGCGAGGACAAGGA | With mismatches (-A) | 90 |
| Mutation type 1 probe 3 | CGAGGAGCGAGGACAA | With mismatches (-A) | 91 |
| Mutation type 1 probe 4 | GACGAGGAGCAGAGGACAAGGAG | Fully matched | 92 |
| Mutation type 1 probe 5 | GACGAGGAGCGAGGACAAGGAG | With mismatches (-A) | 93 |
| Mutation type 1 probe 6 | GACGAGGAGCAGGACAAGGAG | With mismatches (-GA) | 94 |
| Mutation type 1 probe 7 | GACGAGGAGCGGACAAGGAG | With mismatches (-AGA) | 95 |

In this experimentation example, the fluorescence intensity to mutation model samples (5% of mutation plasmids) and the fluorescence intensity to wild-type model samples (plasmids) were measured using the probes shown in Table 8. The results are shown in Table 9. In Table 9, “Specific fluorescence intensity*1” indicates the fluorescence intensity to the mutation model samples and “Non-specific fluorescence intensity*2” indicates the fluorescence intensity to the wild-type model samples.

TABLE 9

| | Probe's | | Specific | Non-specific | |
|---|---|---|---|---|---|
| | nucleotide | Tm | fluorescence | fluorescence | |
| Probe | number | (° C) | intensity*1 | intensity*2 | Remarks |
| Mutation type 1 probe 1 | 17 | 55.0 | 9041 | 3173 | Fully matched |
| Mutation type 1 probe 2 | 17 | 56.8 | 513 | 106 | With mismatches (-A) |
| Mutation type 1 probe 3 | 17 | 56.8 | 296 | 16 | With mismatches (-A) |
| Mutation type 1 probe 4 | 23 | 62.9 | 37343 | 37224 | Fully matched |
| Mutation type 1 probe 5 | 22 | 63.4 | 29012 | 22051 | With mismatches (-A) |
| Mutation type 1 probe 6 | 21 | 61.4 | 23192 | 9976 | With mismatches (-GA) |
| Mutation type 1 probe 7 | 20 | 61.9 | 15166 | 2905 | With mismatches (-AGA) |

As shown in Table 9, a probe having a deletion-type mismatch at a given site was found to be capable of specifically hybridizing to a mutation sample exhibiting high specific fluorescence intensity*1 and low non-specific fluorescence intensity*2. As shown in Table 9, a probe capable of hybridizing specifically to a mutation sample was designed (specific fluorescence intensity*1 of 15,000 or higher and non-specific fluorescence intensity*2 of 3,000 or lower) (the type 1 mutation probe 7).

### Experimentation Example 2

In this experimentation example, a plurality of sets of primers used to amplify a region including 6 gene mutations in exon 12 of JAK2 were designed and evaluated. The designed sets of primers are as shown in FIG. 3. In this example, the sets of primers shown in Table 10 below were evaluated. F1 to F5 and R1 to R3 in Table 10 correspond to FIG. 1. As shown in FIG. 3, the forward primers F1 and F3 to F5 were designed to be included in the nucleotide sequence represented by SEQ ID NO: 1 and the forward primer F2 was designed to be excluded from the nucleotide sequence represented by SEQ ID NO: 1 (i.e., SEQ ID NO: 52). In Experimentation Example 1 and Experimentation Example 2, genome DNA derived from the peripheral blood of a healthy individual was used as a wild-type sample and evaluated based on the fluorescence intensity derived from the wild-type probe.

TABLE 10

| | Forward primer | Reverse primer |
|---|---|---|
| Set of primers 1 | F1 | R1 |
| Set of primers 2 | F2 | R2 |
| Set of primers 3 | F3 | R3 |
| Set of primers 4 | F4 | R3 |
| Set of primers 5 | F5 | R2 |

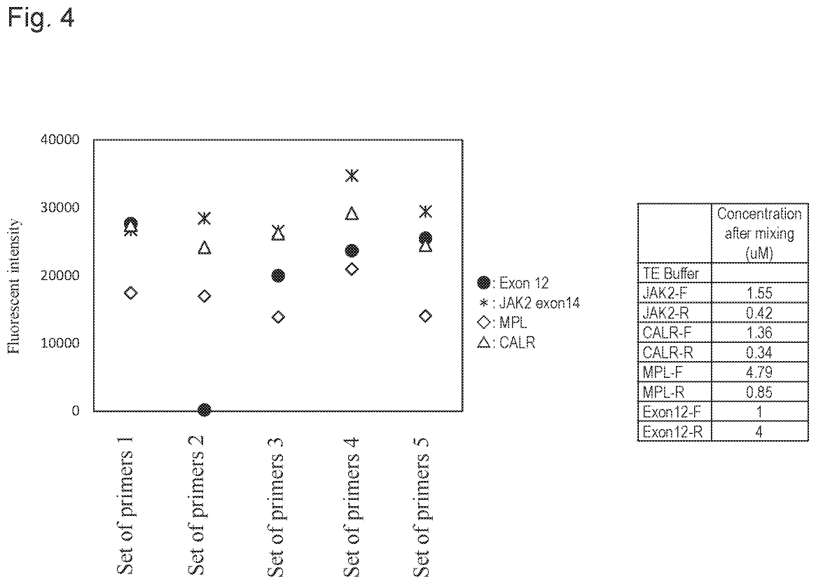

The results are shown in FIG. 4. With the use of a set of primers other than the set of primers 2 using F2 among the sets of primers designed in this example, as is apparent from FIG. 4, satisfactory fluorescence intensities would be achieved for all the amplified fragments. Among such sets of primers, the sets of primers 1, 4, and 5 generally exhibiting high fluorescence intensity may be preferable, and sets of primers 1 and 5 exhibiting relatively small differences in intensities between target regions may be more preferable. In the following evaluation, the set of primers 5 (F5 in combination with R2) was used as shown in Table 2.

### Experimentation Example 3

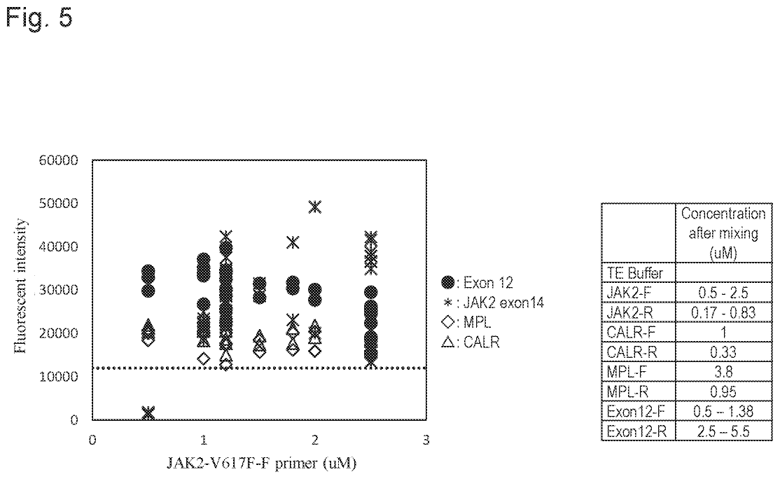

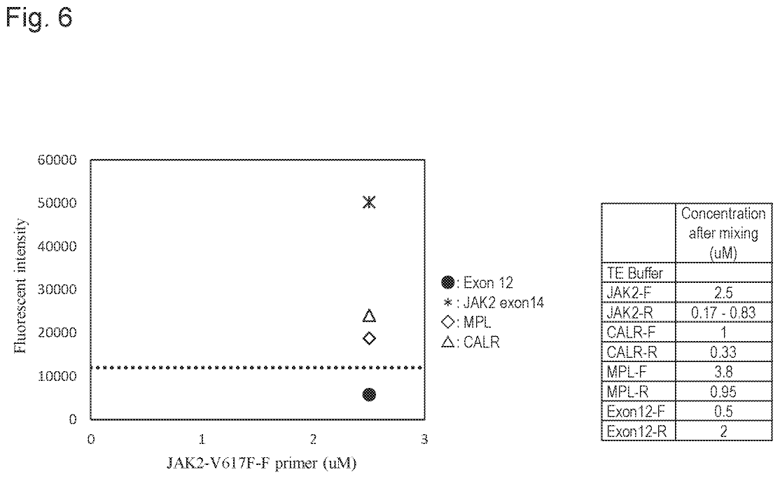

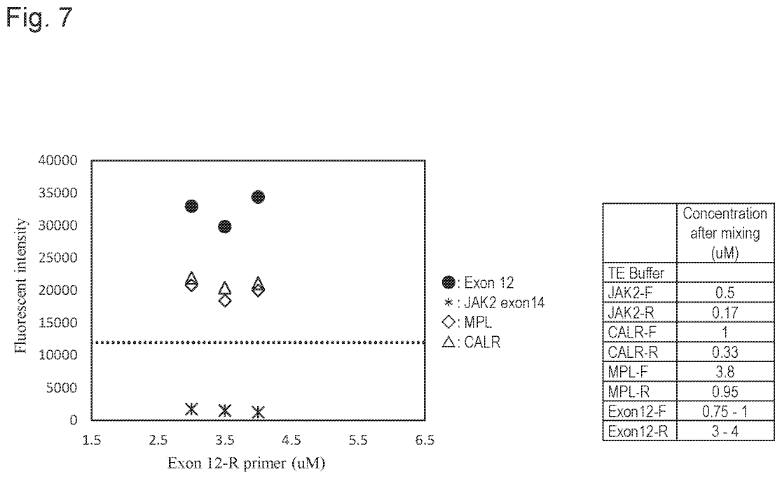

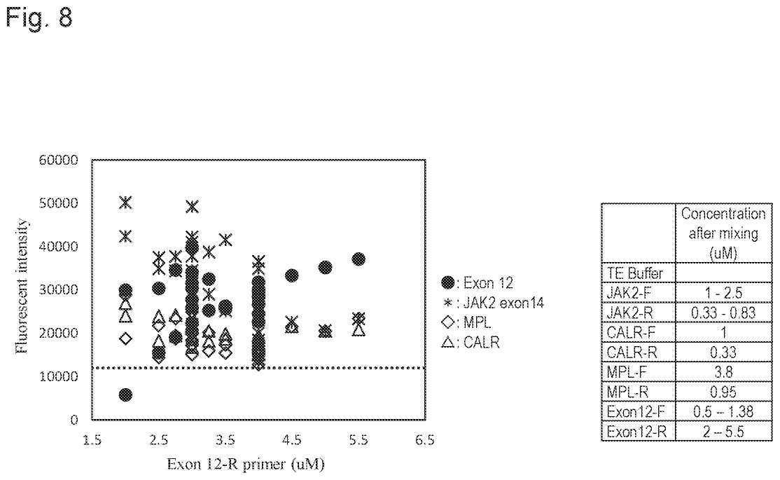

FIG. 5 and FIG. 6 each show a characteristic diagram demonstrating, on the horizontal axis, the concentration of primers mixed in a PCR solution and the concentration of the labeled primer (the forward primer) in the set of primers that amplifies a region including the V617F mutation and, on the vertical axis, the fluorescence intensities derived from the amplified 4 regions. FIG. 7 and FIG. 8 each show a characteristic diagram demonstrating, on the horizontal axis, the concentration of primers mixed in a PCR solution and the concentration of the labeled primer (the reverse primer) in the set of primers that amplifies exon 12 in JAK2 and, on the vertical axis, the fluorescence intensities derived from the amplified 4 regions.

When the concentration of a labeled primer of the set of primers that amplifies the region including the V617F mutation is 0.5 μM, as is apparent from FIG. 5, the fluorescence intensity of 12,000 or higher could not be achieved. When the concentration of a labeled primer of the set of primers that amplifies the region including the V617F mutation is 2.5 μM and the concentration of a labeled primer of the set of primers that amplifies exon 12 is 2.0 μM, as is apparent from FIG. 6, the fluorescence intensity of 12,000 or higher could not be achieved.

When the concentration of a labeled primer of the set of primers that amplifies exon 12 is 3.0 to 4.0 μM and the concentration of a labeled primer of the set of primers that amplifies a region including the V617F mutation is 0.5 μM, as is apparent from FIG. 7, the fluorescence intensity of 12,000 or higher could not be achieved. When the concentration of a labeled primer of the set of primers that amplifies the region including the V617F mutation is 1.0 μM or higher and the concentration of a labeled primer of the set of primers that amplifies exon 12 is 2.5 μM or higher, as is apparent from FIG. 8, the fluorescence intensity of 12,000 or higher was achieved.

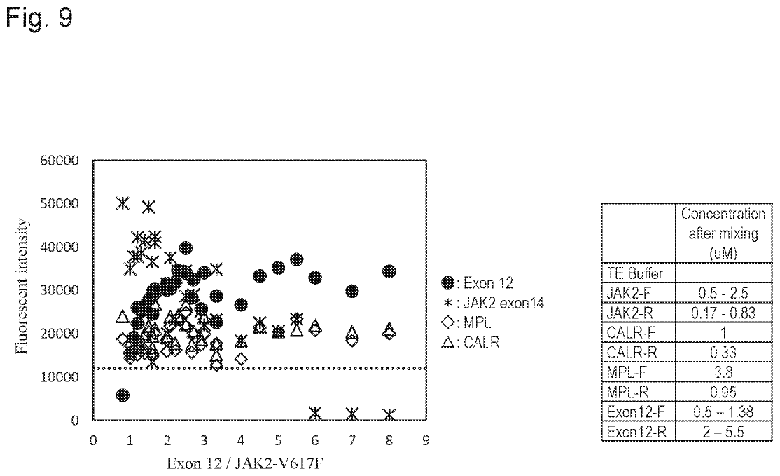

FIG. 9 shows a table demonstrating the concentrations of primers mixed in a PCR solution and a characteristic diagram demonstrating, on the horizontal axis, the ratio of the concentration of the labeled primer (the reverse primer) of the set of primers that amplifies exon 12 to the concentration of the labeled primer (the forward primer) of the set of primers that amplifies a region including the V617F mutation and, on the vertical axis, the fluorescence intensities derived from the amplified 4 regions. When the concentration ratio is between 1.0 and 5.5, as is apparent from FIG. 9, the fluorescence intensity of 12,000 or higher was achieved for all the amplified fragments.

### Example 2

In this example, an artificial gene (plasmid) comprising a wild-type or mutant sequence of the target region was constructed for the mutation model sample, and a mixture of the artificial wild-type gene and the artificial mutant gene at any mixing ratio was subjected to PCR to detect mutation using the reaction composition shown in Table 11.

TABLE 11

| Reagent | Manufacturer | Volume (μl) |
|---|---|---|
| 10 × PCR Buffer | Roche Diagnostics | 4.0 |
| 10 nM dNTP mix | Roche Diagnostics | 0.8 |
| Faststart DNA taq polymerase | Roche Diagnostics | 0.4 |
| Primer mix | Life Technologies Japan | 4.0 |
| DNA sample (0.16 pg/μl) | | 10.0 |
| Purified water | | 20.8 |

In this example, a PCR solution was supplemented with the blocker oligo DNA shown in Table 12. A blocker is added to suppress non-specific hybridization of a probe for mutation detection, so that sufficient detection sensitivity is achieved even when an extent of mutation of the target gene is small. A blocker is designed to specifically hybridize to a wild-type-derived amplification product.

TABLE 12

| Blocker | | SEQ ID |
|---|---|---|
| oligo DNA | Sequence (5′-3′) | NO: |
| JAK2 V617F | CTCCACAGACACATACTC | 31 |
| blocker | | |
| CARL blocker | CCTCCTCCTCTTTGCG | 32 |
| MPL blocker | AAACTGCCACCTCAGC | 33 |
| JAK2 exon12 | CACAAAATCAGAAATGAAGATTTG | 34 |
| blocker | | |

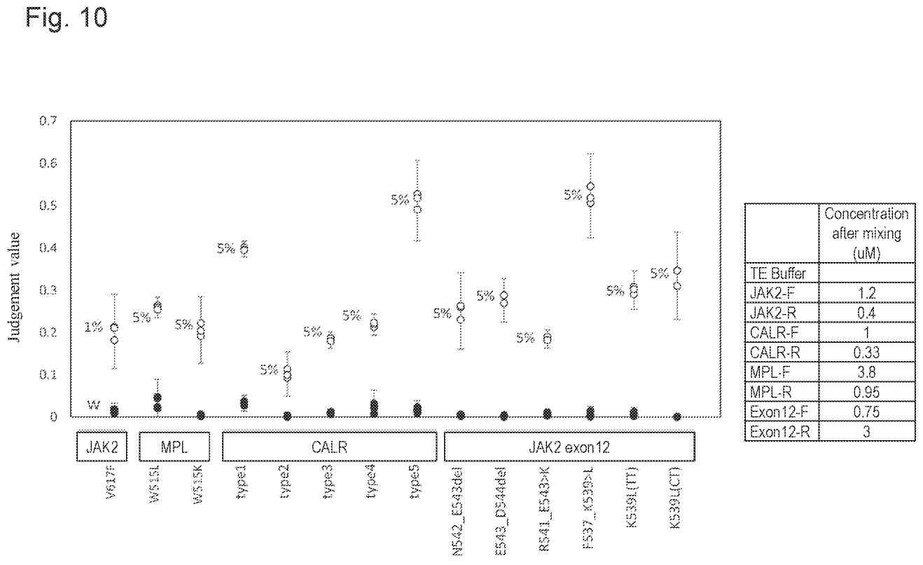

In this example, wild-type samples (n=9) in which the entire target gene region is of a wild type and mutation model samples (n=3) in which a part of the target gene region is a mutant were used, and the percentage of mutation model samples was 1% or 5%. The results are shown in FIG. 10. In FIG. 10, an error bar indicates 5σ.

As shown in FIG. 10, all the mutation model samples accounting for 1% or 5% of the total samples were identified.

All publications, patents, and patent applications cited herein are incorporated herein by reference in their entirety.

1. A kit for evaluating a gene mutation related to myeloproliferative neoplasms comprising a CALR mutation probe corresponding to the gene mutation related to myeloproliferative neoplasms in CALR, which is at least 1 gene mutation selected from the group consisting of: a 52-bp deletion type 1 mutation resulting from deletion of 52 nucleotides at positions 506 to 557 in the nucleotide sequence of the wild-type CALR gene represented by SEQ ID NO: 10; a 46-bp deletion type 3 mutation resulting from deletion of 46 nucleotides at positions 509 to 554 in the nucleotide sequence represented by SEQ ID NO: 10; a 34-bp deletion type 4 mutation resulting from deletion of 34 nucleotides at positions 516 to 549 in the nucleotide sequence represented by SEQ ID NO: 10; and a 52-bp deletion type 5 mutation resulting from deletion of 52 nucleotides at positions 505 to 556 in the nucleotide sequence represented by SEQ ID NO: 10, wherein the CALR mutation probe comprising mismatches caused by artificial deletion.

2. The kit for evaluating a gene mutation according to claim 1, wherein
the CALR mutation probe corresponding to the type 1 mutation comprises a nucleotide sequence derived from the nucleotide sequence represented by SEQ ID NO: 10 by deletion of 1 or a plurality of nucleotides selected from a range of 558 to 564 or a nucleotide sequence complementary thereto,
the CALR mutation probe corresponding to the type 3 mutation comprises a nucleotide sequence derived from the nucleotide sequence represented by SEQ ID NO: 10 by deletion of 1 or a plurality of nucleotides selected from a range of 555 to 559 or a nucleotide sequence complementary thereto,
the CALR mutation probe corresponding to the type 4 mutation comprises a nucleotide sequence derived from the nucleotide sequence represented by SEQ ID NO: 10 by deletion of 1 or a plurality of nucleotides selected from nucleotides 550 to 558 or a nucleotide sequence complementary thereto, and
the CALR mutation probe corresponding to the type 5 mutation comprises a nucleotide sequence derived from the nucleotide sequence represented by SEQ ID NO: 10 by deletion of 1 or a plurality of nucleotides selected from a range of 558 to 564 or a nucleotide sequence complementary thereto.

3. The kit for evaluating a gene mutation according to claim 1, wherein the CALR mutation probe corresponding to the type 1 mutation comprises the nucleotide sequence represented by SEQ ID NO: 95 or a nucleotide sequence complementary thereto, the CALR mutation probe corresponding to the type 3 mutation comprises the nucleotide sequence represented by SEQ ID NO: 53 or a nucleotide sequence complementary thereto, the CALR mutation probe corresponding to the type 4 mutation comprises the nucleotide sequence represented by SEQ ID NO: 54 or a nucleotide sequence complementary thereto, and the CALR mutation probe corresponding to the type 5 mutation comprises the nucleotide sequence represented by SEQ ID NO: 55 or a nucleotide sequence complementary thereto.

4. The kit for evaluating a gene mutation according to claim 1, which further comprises a CALR mutation probe corresponding to the type 2 mutation resulting from insertion of TTGTC between positions 568 and 569 in the nucleotide sequence of the wild-type CALR gene represented by SEQ ID NO: 10.

5. The kit for evaluating a gene mutation according to claim 1, which further comprises a JAK2 mutation probe corresponding to the gene mutation related to myeloproliferative neoplasms in JAK2 and/or an MPL mutation probe corresponding to the gene mutation related to myeloproliferative neoplasms in MPL.

6. A method of analyzing data concerning diagnosis of myeloproliferative neoplasms, comprising identifying at least 1 gene mutation selected from the group consisting of the type 1 mutation, the type 3 mutation, the type 4 mutation, and the type 5 mutation related to myeloproliferative neoplasms in CALR using the kit for evaluating a gene mutation according to claim 1 in a target of diagnosis.

7. A kit for evaluating a gene mutation related to myeloproliferative neoplasms comprising JAK2 mutation probes corresponding to the gene mutation related to myeloproliferative neoplasms in JAK2 and a set of primers that amplifies a region including the gene mutation, wherein the JAK2 mutation probes comprise a V617F mutation probe corresponding to the V617F mutation and an exon 12 mutation probe corresponding to a gene mutation existing in exon 12 of the JAK2 gene, and the set of primers comprises a set of primers for the V617F mutation that amplifies a region including the V617F mutation and a set of primers for exon 12 that amplifies a region including the gene mutation existing in exon 12 of the JAK2 gene.

8. The kit for evaluating a gene mutation according to claim 7, wherein the exon 12 mutation probe is at least 1 mutation probe selected from the group consisting of a N542_E543del mutation probe corresponding to a deletion mutation of N542-E543 in JAK2, a E543_D544del mutation probe corresponding to a deletion mutation of E543-D544 in JAK2, a R541_E543>K mutation probe corresponding to a substitution mutation of R541-E543 with lysine in JAK2, a F537_K539>L mutation probe corresponding to a substitution mutation of F537-K539 with leucine in JAK2, a K539L (TT) mutation probe corresponding to a mutation of K539L (TT) in JAK2, and a K539L (CT) mutation probe corresponding to a mutation of K539L (CT) in JAK2.

9. The kit for evaluating a gene mutation according to claim 7, wherein the concentration of a primer included in the set of primers for the V617F mutation is 1.0 μM or higher.

10. The kit for evaluating a gene mutation according to claim 7, wherein the concentration of a primer included in the set of primers for exon 12 is 2.5 μM or higher.

11. The kit for evaluating a gene mutation according to claim 7, wherein the ratio of the concentration of the labeled primer of the set of primers for the V617F mutation to the concentration of the labeled primer of the set of primers for exon 12; the concentration of the primer for exon 12/the concentration of the primer for the V617F mutation is 1.0 to 5.5.

12. The kit for evaluating a gene mutation according to claim 7, wherein the set of primers for exon 12 consists of a forward primer for exon 12 having 10 or more continuous nucleotides selected from the nucleotide sequence represented by SEQ ID NO: 1 and reverse primer for exon 12 having 10 or more continuous nucleotides selected from the nucleotide sequence represented by SEQ ID NO: 2.

13. The kit for evaluating a gene mutation according to claim 12, wherein the forward primer for exon 12 is a primer selected from the group consisting of a forward primer F1 for exon 12 comprising the nucleotide sequence represented by SEQ ID NO: 3, a forward primer F3 for exon 12 comprising the nucleotide sequence represented by SEQ ID NO: 4, a forward primer F4 for exon 12 comprising the nucleotide sequence represented by SEQ ID NO: 5, and a forward primer F5 for exon 12 comprising the nucleotide sequence represented by SEQ ID NO: 6.

14. The kit for evaluating a gene mutation according to claim 12, wherein the reverse primer for exon 12 is a primer selected from the group consisting of a reverse primer R1 for exon 12 comprising the nucleotide sequence represented by SEQ ID NO: 7, a reverse primer R2 for exon 12 comprising the nucleotide sequence represented by SEQ ID NO: 8, and a reverse primer R3 for exon 12 comprising the nucleotide sequence represented by SEQ ID NO: 9.

15. The kit for evaluating a gene mutation according to claim 12, wherein the set of primers for exon 12 consists of the forward primer F5 for exon 12 comprising the nucleotide sequence represented by SEQ ID NO: 6 and the reverse primer R2 for exon 12 comprising the nucleotide sequence represented by SEQ ID NO: 8.

16. The kit for evaluating a gene mutation according to claim 7, which further comprises:
a CALR mutation probe corresponding to the gene mutation related to myeloproliferative neoplasms in CALR;
a set of primers for CALR for amplifying a region including the gene mutation related to myeloproliferative neoplasms in CALR;
an MPL mutation probe corresponding to the gene mutation related to myeloproliferative neoplasms in MPL; and
a set of primers for MPL for amplifying a region including the gene mutation related to myeloproliferative neoplasms in MPL.

17. The kit for evaluating a gene mutation according to claim 7, which comprises a microarray having the V617F mutation probe and the exon 12 mutation probe fixed on a support.

18. A method of analyzing data concerning diagnosis of myeloproliferative neoplasms, comprising simultaneously identifying the V617F mutation and the gene mutation in exon 12 from among the JAK2 gene mutations related to myeloproliferative neoplasms using the kit for evaluating a gene mutation according to claim 7 in a target of diagnosis.
